SEQUENCE LISTING (12 sequences)

SEQ ID NO: 1 - protein, 278 aa, Brevibacillus borstelensis
MREPFVLEGEKSILALADWQAHFPGLVAGFTVRLGGESEEPYGSFNMGLHVGDDPAHVIANRKKLAEQVGMPFEAWTCADQVHGNRVCQVTAGGAGKESLGDVIAATDGLFTQQKGVLLTSFYADCVPLYFLDPASGAIGLAHAGWKGTVGRIAEEMVKALQTHYKAKPGDIRIAIGPSIGGCCYEVDERIMTQVRTSAENWKTAVSASTEGKYMLDLRQLNTEILREAGISRANMLVTDWCTSCRTDLFFSHRKEAGPGKMTGRMASYIGWKETEGR

SEQ ID NO: 2 - DNA, 1623 bp, Brevibacillus agri
atgaacaaatcatcgttacgaagcacagccttcccgcttttgctgggcggtctgctgcttctgtccgcctgctcgaccgagcaagcgacgaccgcgggccacgccgggcacgacatgggagccgaccaaagcgcgacgcagcaaccggctgctccctcccaaccgatgactgcgtcaggcgacaatgccatggaggtgctgacgggcaatacgttcaccctcacggcaaaagagagcatgctgcacctcgacgaccagacgatgaaaacagcctggacctacaacggaaccgtccctggaccgcagcttcgcgtcaagcagggcgagacgatttccgtcaccttgaaaaatgaactgccggagccggtgacgatccactggcacgggctgcctgtgccaaacaacatggacggcatccccggtgtcacgcaaaatgcggtgaagccaaacgaaagcttcacctaccgcttcaaggtcgacgtggcgggaacgtactggtaccactcgcatcaaaacagctccaggcaggtcgacaaagggctgtacggctcgctcgtcgtcgagccgaaaacgccggagccagcagacaaagacgtcacgctcgtcctcgacgaatggatgcaggacgacagcatggccgaaatgcacggtggcggcggctcgatggcaggcatgaaccacggtgctgaccacgccgctcccgccacctctgctgcgagcggccacgacatggcgaacatgagcgacgcgaaaatgatgccgctcatgtacacgatcttttccgtcaacgggaagacgggacctgccatcgctccgctgcgcgtgaaggaaggcgaaaaagtccgcatccgcctcatcaatgccgggtatttgtcgcacaagctgaacctgcaaggacatgcgttccaaatcgtttccacggacgggcagccgctgcacaatccgccgctcacgagcggacagttgctcaacatcgcccccggcgagcgctacgatctcgaatttgtagcgaacaacccgggaacatggctgctggaggagcgaagcgacaaccctggcgccaaatcgcttgccgtgcctatcgtctacgaaggctacgaagcggcgcaggccaaaccggagtcgggtcaactcccggtcattgatctcacccgatacggcgaagcggcccaaagcagcttttcgctggagcagccgtacgatatcacataccgaatggacttgaacaccgactcgcgcgacgggcagatggtgtttacgatcaacggccaaacgttcccgaacgtccctccgctggatgtaaaaaaaggcgaccgggtcaaggtgaccatcgtcaacaactcgccggaggacgtccacccgatgcatttgcacggacacttcttccaggtgctgagcaaaaacggccagcccgtgtccggctcgccgctggtcaaggacaccttgaatgtgctgccaggcgagtcctacgtcgtcgcctttgcggctgacaatcccggcgagtggatgttccactgccacgacctggggcatgcggccaaagggatggtgtccgaggtcaaatacacgggcttccagcgggacttcgtcgtcgatccgaccgtcggcaacatgccggagtaa

SEQ ID NO: 3 - protein, 540 aa, Brevibacillus agri
MNKSSLRSTAFPLLLGGLLLLSACSTEQATTAGHAGHDMGADQSATQQPAAPSQPMTASGDNAMEVLTGNTFTLTAKESMLHLDDQTMKTAWTYNGTVPGPQLRVKQGETISVTLKNELPEPVTIHWHGLPVPNNMDGIPGVTQNAVKPNESFTYRFKVDVAGTYWYHSHQNSSRQVDKGLYGSLVVEPKTPEPADKDVTLVLDEWMQDDSMAEMHGGGGSMAGMNHGADHAAPATSAASGHDMANMSDAKMMPLMYTIFSVNGKTGPAIAPLRVKEGEKVRIRLINAGYLSHKLNLQGHAFQIVSTDGQPLHNPPLTSGQLLNIAPGERYDLEFVANNPGTWLLEERSDNPGAKSLAVPIVYEGYEAAQAKPESGQLPVIDLTRYGEAAQSSFSLEQPYDITYRMDLNTDSRDGQMVFTINGQTFPNVPPLDVKKGDRVKVTIVNNSPEDVHPMHLHGHFFQVLSKNGQPVSGSPLVKDTLNVLPGESYVVAFAADNPGEWMFHCHDLGHAAKGMVSEVKYTGFQRDFVVDPTVGNMPE

SEQ ID NO: 4 - DNA, 23 bp, Artificial Sequence
  Primer
atgaacaaatcatcgttacgaag

SEQ ID NO: 5 - DNA, 23 bp, Artificial Sequence
  Primer
ttactccggcatgttgccgacgg

SEQ ID NO: 6 - DNA, 24 bp, Artificial Sequence
  Primer
tcattttcgcgtcgctcatgttcg

SEQ ID NO: 7 - DNA, 24 bp, Artificial Sequence
  Primer
aatgtgctgccaggcgagtcctac

SEQ ID NO: 8 - DNA, 38 bp, Artificial Sequence
  Primer
atgctacaattgatatgaacaaatcatcgttacgaagc

SEQ ID NO: 9 - DNA, 36 bp, Artificial Sequence
  Primer
tagctaactagtatgaacaaatcatcgttacgaagc

SEQ ID NO: 10 - DNA, 38 bp, Artificial Sequence
  Primer
atgtcatgcggccgcctccggcatgttgccgacggtcg

SEQ ID NO: 11 - DNA, 34 bp, Artificial Sequence
  Primer
atcgtaccatatgaacaaatcatcgttacgaagc

SEQ ID NO: 12 - DNA, 40 bp, Artificial Sequence
  Primer
atgctacctcgagtttactccggcatgttgccgacggtcg

(19) United States
(12) Patent Application Publication
(10) Pub. No.: US 2016/0053070 A1
(43) Pub. Date: Feb. 25, 2016

(54) COMPOSITIONS AND METHODS FOR BIODEGRADING PLASTIC

(71) Applicant: B.G NEGEV TECHNOLOGIES LTD., Beer Sheva (IL)

(72) Inventors: Alex Sivan, Meitar (IL); Amir Aharoni, Haifa (IL); Or Press, Lehavim (IL)

(73) Assignee: B.G NEGEV TECHNOLOGIES LTD., Beer Sheva (IL)

(21) Appl. No.: 14/783,390

(22) PCT Filed: Apr. 6, 2014
(86) PCT No.: PCT/IL2014/050332

§ 371 Date: Oct. 8, 2015

Related U.S. Application Data
(60) Provisional application No. 61/810,890, filed on Apr. 11, 2013.
(60) Provisional application No. 61/847,616, filed on Jul. 18, 2013.

(51) Int. Cl. C08J 11/10; C12N 9/02

(52) U.S. Cl. CPC C08J 11/105; C08J 11/10; C12N 9/0061; C08J 2323/06; C12Y 110/03002

(57) ABSTRACT

The present invention provides a composition including polyethylene and laccase wherein the laccase has an optimal specific activity at a temperature of 60° C. to 100° C. and/or in the presence of xylan. Furthermore, the invention covers a method for biodegrading/decomposing plastic by contacting laccase with an optimal specific activity at a temperature of 60° C. to 100° C. with plastic or contacting a microorganism expressing a laccase with an optimal specific activity at a temperature of 60° C. to 100° C. with plastic.

### FIELD OF INVENTION

This invention is directed to; inter alia, compositions based on thermophilic laccase and to methods utilizing the thermophilic laccase or a microorganism expressing it for decomposing/biodegrading plastic.

### BACKGROUND OF THE INVENTION

Human industrial activities inevitably generate industrial wastes. These industrial wastes primarily consist of inorganic and organic waste discharged by factories, agriculture, fisheries and food processing industries. The high cost of biodegrading or handling these wastes are borne by these industries. These costs hinder market expansion for these and other related businesses.

Currently, organic waste fermentation and treatment systems have been developed for utilizing waste. Using these systems, one can currently produce biologically active substances such as soil improvement agents, and compost.

Plastics provide a number of benefits because they are generally lighter, stronger, more durable, and more resistant to water. The same properties that make traditional plastics an ideal material for many uses, however, also tend to cause environmental problems at the end of the useful life of these materials as the inherent strength and durability of these materials allow them to persist in the environment without biodegrading.

After consumer use, plastic bags frequently end up as litter in the environment or in landfills. Because traditional plastics are not biodegradable, discarded plastics represent a significant environmental problem in either place. As litter, plastics are a visible and widespread pollutant, a threat to animal and marine species, and to human health. In landfills, plastic bags add to landfill volume, hinder landfill compaction and delay the biodegradation of discarded organic materials trapped inside, thereby fostering the formation of methane, a harmful greenhouse gas.

In light of global environmental protection, the recent most important issue is to construct circulating social systems that can be maintained and continued. In such a social situation, much effort has also been made to develop recycling techniques for plastic wastes. Recycling techniques for plastic wastes are divided into two major types: physical approach (thermal recycling, material recycling) and chemical approach (chemical recycling). Among them, the former physical treatment has already been made practical for PET or other resins on a commercial basis because it is relatively simple and cost-effective. However, this approach cannot avoid quality loss due to repeated use and hence the resulting recycled products will have limited applications.

On the other hand, in the case of chemical recycling, plastic waste matrials are defragmented into monomers or oligomers, which are then collected and used as source materials for resynthesis of new plastics. This approach allows production of plastic products completely comparable to primary products, and causes no quality loss. In light of these facts, products with chemical recycling in mind have recently been developed and a part of them has already been on the market.

In the future, biodegradable plastics will constitute nearly half of the total yield of plastics, and it is also expected that attention will be given to the development of efficient techniques for their recycling in the future. Biodegradable plastics currently in circulation are almost exclusively polyester-based plastics. This means that monomer recycling can be very easily achieved for these plastics because their monomer components such as organic acids and polyhydric alcohols are joined via ester bonds sensitive to hydrolysis.

However, a large problem arises when actually attempting recycling because wastes comprise multiple types of materials in admixture. Although there is a cry for separated collection of wastes, it will actually be impossible to completely achieve separated collection when taking into account the awareness of people who discard wastes as well as the time and effort required for separated collection. In particular, plastic products generally use a plurality of different plastics in combination, and currently used techniques do not enable the separation of all plastic wastes according to plastic types. For this reason, under present circumstances, recycling is limited to wastes which are easy to separate and collect, regardless of recycling techniques.

To overcome this problem, a new process has been proposed in which enzymes are used for chemical degradation of plastics. As to merits resulting from the use of enzymes, the substrate specificity of enzymes may be an excellent feature although it is also important in that reactions can be carried out at normal temperature and under normal pressure, thereby saving energy costs and requiring no organic solvent responsible for environmental pollution. In general, enzymes have substrate specificity and clearly select their target substrates. Thus, a combination of enzymes, each being reactive to only a certain specific plastic, allows efficient extraction of high purity monomers from plastic wastes in a mixture form, without requiring any separation process. In general, bioprocesses require high costs and hence are disadvantageous in this point without any doubt, but it is a great merit to achieve extraction of high purity monomers without separation. Particularly, also in view of the fact that biodegradable plastics are degraded by the action of enzymes secreted by microorganisms in the natural world, it can be expected to develop a process using enzymes derived from such biodegrading bacteria.

To establish enzymatic recycling, the premise is the presence of a strong plastic-biodegrading enzyme having high substrate specificity. In particular, since plastic wastes are practically discarded in solid form such as chips and/or blocks, bacteria which degrade solids are particularly important.

Examples known as enzymes derived from unnatural plastic-biodegrading bacteria are those capable of biodegrading ester-based polyurethanes. Such enzymes are derived from Comamonas acidovorans and cleave ester bonds in ester-based solid polyurethanes to produce water-soluble monomers [Akutsu, Y., Nakajima-Kambe, T., Nomura, N., and Nakahara, T.: Purification and properties of a polyester polyurethane-biodegrading enzyme from C. acidovorans TB-35. Appl. Environ. Microbiol., 64, 62-67 (1998); and JP 09-224664 A entitled “Method for polyurethane esterase purification and method for ester-based polyurethane degradation” (Applicant: Suzuki Motor Corporation; Inventors: Toshiaki Nakajima, et al.)].

Although there have been many reports of biodegrading bacteria for these biodegradable plastics, most of these reports were directed degradation of emulsified or powdered plastics or thin films of micron order (Kim, D. Y., and Rhee, Y. H.: Biodegradation of microbial and synthetic polyesters by fungi. Appl. Microbiol. Biotechnol., 61, 300-308 (2003)). Uchida et al. have isolated Acidovolax delafieldii strain BS-3 which assimilates PBSA pellets as a sole carbon source [JP 11-225755 A entitled “Biodegradable polymer-biodegrading enzyme and method for its preparation” (Applicant: Mitsubishi Chemical Corporation; Inventors: Toshiaki Nakajima, et al.); N., Tokiwa, Y., and Nakahara, T.: Properties of a bacterium which degrades solid poly(tetramethylene succinate)-co-adipate, a biodegradable plastic. FEMS Microbiology Letters, 189, 25-29, (2000)], but there is no other report about bacteria capable of biodegrading solid pellets.

In view of the foregoing, there are a limited number of reports on microorganisms capable of biodegrading plastics in film or pellet form, and further their enzymes are poorly known. To establish enzymatic recycling, there is a strong demand for enzymes capable of rapidly biodegrading solid plastics.

### SUMMARY OF THE INVENTION

In one embodiment, the present invention provides a composition comprising: polyethylene and laccase, wherein the laccase has an optimal specific activity at a temperature of 60° C. to 100° C. In another embodiment, the laccase comprises the amino acid sequence of SEQ ID NO: 1. In another embodiment, the laccase comprises the amino acid sequence of SEQ ID NO: 3. In another embodiment, the laccase is B. borstelensis laccase. In another embodiment, the laccase is B. agri laccase.

In another embodiment, the present invention further provides a method for decomposing/biodegrading polyethylene, comprising the step of contacting polyethylene with a laccase having an optimal specific activity at a temperature of 60° C. to 100° C. In another embodiment, the method includes maintaining the reaction temperature at 60° C. to 100° C.

In another embodiment, the present invention further provides a method for decomposing polyethylene, comprising the step of contacting polyethylene with Brevibacillus borstelensis, Brevibacillus agri, or a combination of B. borstelensis and B. agri, wherein the polyethylene is the only carbon source for the bacteria. In another embodiment, the method further includes maintaining the polyethylene and the Brevibacillus bacteria at a temperature of 35° C. to 50° C.

### BRIEF DESCRIPTION OF THE DRAWINGS

FIG. 1. A graph showing the optimal temperature growth conditions for Brevibacillus borstelensis and Brevibacillus agri.

FIG. 2. A graph showing the optimal pH growth conditions for Brevibacillus borstelensis and Brevibacillus agri.

FIG. 3. A graph showing the optimal temperature conditions for the activity of laccase isolated from Brevibacillus borstelensis and for the activity of laccase isolated from Brevibacillus agri.

FIG. 4. A bar graph showing the effect of copper concentration on Laccase specific activity in B. borstelensis.

FIG. 5. A bar graph showing the effect of copper concentration on Laccase concentration in B. borstelensis.

FIG. 6. A bar graph showing the effect of copper concentration on Laccase specific activity in B. agri.

FIG. 7. A bar graph showing the effect of copper concentration on Laccase concentration in B. agri

FIG. 8. A bar graph showing that B.agri had a better polyethylene biodegradation ability compared to Brevibacillus borstelensis.

FIG. 9. SEM micrographs (×10,000) showing laccase biodegraded polyethylene (A) and control (B).

FIG. 10. A bar graph showing the effect of xylan and/or copper on the polyethylene digestion efficiency of laccase of Rhodococcus ruber bacteria after 7 days incubation. Control included copper, xylan and polyethylene without a laccase or a bacteria comprising it.

FIG. 11. 16SrRna sequence Brevibacillus agri.

FIG. 12. Phylogenetic tree of both strains based on their 16SrRNA sequence with rhodococcus ruber as an external strain.

FIG. 13. Bacterial growth curve in 40□ C of Brevibacillus agri.

FIG. 14. Laccase excretion to the bacterial extracellular medium at 00:00 hours

FIG. 15. Laccase excretion to the bacterial extracellular medium at 19:40 hours.

FIG. 16. Excreted laccase activity in different measurement times.

FIG. 17. Laccase relative activity in different temperatures. All experiment were made with the same enzyme concentration.

FIG. 18. Laccase activity in different temperatures after 30 and 90 minutes.

FIG. 19. The effect of ABTS on Laccase induction.

FIG. 20. The effect of ABTS on Laccase specific activity.

FIG. 21. The effect of xylan on (A) Laccase induction, and (B) Laccase specific activity.

FIG. 22. The effect of xylan and copper synergism on (A) Laccase induction, and (B) Laccase specific activity, xylan concentration of 100 μg/mL.

FIG. 23. The effect of cobalt on (A) Laccase induction, and (B) Laccase specific activity.

FIG. 24. The effect of Nickel on (A) Laccase induction, and (B) Laccase specific activity.

FIG. 25. Comparison between the effects of the different additives on Laccase activity.

FIG. 26. FTIR analysis of PE samples after 30 days biodegradation experiment in the presence of different additives. (A) FTIR spectrum, (B) Carbonyl index for each treatment.

FIG. 27. DSC analysis of PE samples after 30 days biodegradation experiment in the presence of different additives (A) DSC curve, (B) Parameters received from DSC analysis.

FIG. 28. FTIR analysis of PE samples after 30 days biodegradation experiment in the presence of different additives preformed after a pre-incubation with laccase enzyme for 7 days. (A) FTIR spectrum, (B) Carbonyl index for each treatment.

FIG. 29. DSC analysis of PE samples after 30 days biodegradation experiment in the presence of different additives preformed after a pre-incubation with laccase enzyme for 7 days. (A) DSC curve, (B) Parameters received from DSC analysis

FIG. 30. Comparison of DSC curves with and without pre-incubation with laccase. (A) DSC curves (B) Parameters received from DSC analysis.

FIG. 31. FTIR analysis of PE incubated with laccase during different periods (A) FTIR spectrum, (B) Carbonyl index for each treatment.

FIG. 32. Comparison of DSC curves of PE samples after different incubation times with laccase. (A) DSC curves (B) Parameters received from DSC analysis.

FIG. 33. SEM images of PE samples. (A) PE sample with no treatment (B) PE sample after 7 days incubation with no enzyme (C) PE sample after 7 days incubation with laccase.

FIG. 34. Multi-copper oxidase sequence from Brevibacillus agri.

FIG. 35. 1% Agarose gel with PCR products of the amplified gene.

FIG. 36. 1% Agarose gel with PCR products from the colony PCR.

FIG. 37. 10% SDS page, with induction products.

FIG. 38. Enzyme induction in different temperatures.

FIG. 39. The effect of copper concentration in bacterial induction medium on laccase activity.

FIG. 40: The effect of copper concentration in the wash and elution buffer on purified laccase activity.

FIG. 41: 10% SDS page with purified protein sample which were cleaned with different copper concentrations in the elution buffer.

FIG. 42: 10% SDS page with monoQ fractions.

FIG. 43: The effect of ABTS concentration on laccase activity (protein stock concentration 0.5 mg/mL).

FIG. 44: The effect of reaction temperature on laccase activity (protein concentration 0.5 mg/mL).

FIG. 45: The effect of temperature on laccase survival.

### DETAILED DESCRIPTION OF THE INVENTION

In one embodiment, the present invention provides a composition comprising of: polyethylene and laccase. In another embodiment, laccase is an oxidase enzyme. In another embodiment, laccase is an oxidase enzyme comprising copper. In another embodiment, laccase is an oxidase enzyme which acts on phenols and similar molecules, performing a one-electron oxidations. In another embodiment, laccase is a bacterial laccase. In another embodiment, laccase is a plant laccase. In another embodiment, laccase is a fungal laccase. In another embodiment, laccase is a thermophilic laccase. In another embodiment, laccase is a thermophilic bacteria laccase. In another embodiment, laccase is an aerobic bacteria laccase. In another embodiment, laccase is a bacterial isolated laccase. In another embodiment, laccase is a bacterial purified laccase. In another embodiment, laccase is a Brevibacillus borstelensis laccase. In another embodiment, laccase is a Brevibacillus agri laccase. In another embodiment, laccase is an extra-cellular bacterial laccase. In another embodiment, laccase is a Rhodococcus ruber laccase. In another embodiment, laccase is a Rhodococcus ruber C208 laccase.

In another embodiment, a composition of the invention is maintained at a temperature of 50° C. to 100° C. In another embodiment, a composition of the invention is maintained at a temperature ranging of 60° C. to 100° C. In another embodiment, a composition of the invention is maintained at a temperature ranging from 70° C. to 90° C. In another embodiment, a composition of the invention is maintained at a temperature ranging from 75° to 85° C. In another embodiment, a composition of the invention is maintained at a temperature ranging from 77° C. to 83° C.

In another embodiment, a laccase has of the present invention has optimal specific activity at a temperature ranging from 50° C. to 100° C. In another embodiment, a laccase of the present invention has an optimal specific activity at a temperature range of 60° C. to 100° C. In another embodiment, a laccase has of the present invention has optimal specific activity at a temperature range of 70° C. to 90° C. In another embodiment, a laccase has of the present invention has optimal specific activity at a temperature range of 75° C. to 85° C. In another embodiment, a laccase has of the present invention has optimal specific activity at a temperature of 77° C. to 83° C.

In another embodiment, a laccase as described herein comprises the amino acid sequence: mrepfvlegeksilaladwqahfpglvagftvrlggeseepygsfnmglhvgddpahvianrkklaeqvgmpfeawtcadqvhgnr vcqvtaggagkeslgdviaatdglftqqkgvlltsfyadcvplyfldpasgaiglahagwkgtvgriaeemvkalqthykakpgdiriaig psiggccyevderimtqvrtsaenwktavsastegkymldlrqlnteilreagisranmlvtdwctscrtdlffshrkeagpgkmtgrma syigwketegr (SEQ ID NO: 1). In another embodiment, a laccase as described herein comprises the amino acid sequence as set forth in SEQ ID NO: 3.

In another embodiment, a laccase as described herein comprises an active fragment of SEQ ID NO: 1 or SEQ ID NO: 3. In another embodiment, an active fragment of a laccase comprises laccase activity. In another embodiment, an active fragment of a laccase comprises polyethylene biodegrading and/or decomposition activity. In another embodiment, an active fragment of a laccase comprises optimal polyethylene biodegrading and/or decomposition activity at a temperature in the range of 60° C. to 100° C. or any other range provided hereinabove for laccase.

In another embodiment, a laccase is a variant of the laccase of SEQ ID NO: 1 which differs from the laccase of SEQ ID NO: 1 by 1-5 conservative amino acid substitutions. In another embodiment, the laccase of the present invention is at least 70% homologous to the laccase of SEQ ID NO: 1 or a peptide thereof. In another embodiment, the amino acid sequence of the laccase of the present invention is at least 75% homologous to the laccase of SEQ ID NO: 1. In another embodiment, the amino acid sequence of the laccase of the present invention is at least 80% homologous to the laccase of SEQ ID NO: 1. In another embodiment, the amino acid sequence of the laccase of the present invention is at least 85% homologous to the laccase of SEQ ID NO: 1. In another embodiment, the amino acid sequence of the laccase of the present invention is at least 90% homologous to the laccase of SEQ ID NO: 1. In another embodiment, the amino acid sequence of the laccase of the present invention is at least 95% homologous to the laccase of SEQ ID NO: 1.

In another embodiment, a laccase is a variant of the laccase of SEQ ID NO: 3 which differs from the laccase of SEQ ID NO: 3 by 1-5 conservative amino acid substitutions. In another embodiment, the laccase of the present invention is at least 70% homologous to the laccase of SEQ ID NO: 3 or a peptide thereof. In another embodiment, the amino acid sequence of the laccase of the present invention is at least 75% homologous to the laccase of SEQ ID NO: 3. In another embodiment, the amino acid sequence of the laccase of the present invention is at least 80% homologous to the laccase of SEQ ID NO: 3. In another embodiment, the amino acid sequence of the laccase of the present invention is at least 85% homologous to the laccase of SEQ ID NO: 3. In another embodiment, the amino acid sequence of the laccase of the present invention is at least 90% homologous to the laccase of SEQ ID NO: 3. In another embodiment, the amino acid sequence of the laccase of the present invention is at least 95% homologous to the laccase of SEQ ID NO: 3.

In another embodiment, a laccase of the invention is encoded by the DNA sequence:

(SEQ ID NO: 2)
ATGAACAAATCATCGTTACGAAGCACAGCCTTCCCGCTTTTGCTGGGCGG
TCTGCTGCTTCTGTCCGCCTGCTCGACCGAGCAAGCGACGACCGCGGGCC
ACGCCGGGCACGACATGGGAGCCGACCAAAGCGCGACGCAGCAACCGGCT
GCTCCCTCCCAACCGATGACTGCGTCAGGCGACAATGCCATGGAGGTGCT
GACGGGCAATACGTTCACCCTCACGGCAAAAGAGAGCATGCTGCACCTCG
ACGACCAGACGATGAAAACAGCCTGGACCTACAACGGAACCGTCCCTGGA
CCGCAGCTTCGCGTCAAGCAGGGCGAGACGATTTCCGTCACCTTGAAAAA
TGAACTGCCGGAGCCGGTGACGATCCACTGGCACGGGCTGCCTGTGCCAA
ACAACATGGACGGCATCCCCGGTGTCACGCAAAATGCGGTGAAGCCAAAC
GAAAGCTTCACCTACCGCTTCAAGGTCGACGTGGCGGGAACGTACTGGTA
CCACTCGCATCAAAACAGCTCCAGGCAGGTCGACAAAGGGCTGTACGGCT
CGCTCGTCGTCGAGCCGAAAACGCCGGAGCCAGCAGACAAAGACGTCACG
CTCGTCCTCGACGAATGGATGCAGGACGACAGCATGGCCGAAATGCACGG
TGGCGGCGGCTCGATGGCAGGCATGAACCACGGTGCTGACCACGCCGCTC
CCGCCACCTCTGCTGCGAGCGGCCACGACATGGCGAACATGAGCGACGCG
AAAATGATGCCGCTCATGTACACGATCTTTTCCGTCAACGGGAAGACGGG
ACCTGCCATCGCTCCGCTGCGCGTGAAGGAAGGCGAAAAAGTCCGCATCC
GCCTCATCAATGCCGGGTATTTGTCGCACAAGCTGAACCTGCAAGGACAT
GCGTTCCAAATCGTTTCCACGGACGGGCAGCCGCTGCACAATCCGCCGCT
CACGAGCGGACAGTTGCTCAACATCGCCCCCGGCGAGCGCTACGATCTCG
AATTTGTAGCGAACAACCCGGGAACATGGCTGCTGGAGGAGCGAAGCGAC
AACCCTGGCGCCAAATCGCTTGCCGTGCCTATCGTCTACGAAGGCTACGA
AGCGGCGCAGGCCAAACCGGAGTCGGGTCAACTCCCGGTCATTGATCTCA
CCCGATACGGCGAAGCGGCCCAAAGCAGCTTTTCGCTGGAGCAGCCGTAC
GATATCACATACCGAATGGACTTGAACACCGACTCGCGCGACGGGCAGAT
GGTGTTTACGATCAACGGCCAAACGTTCCCGAACGTCCCTCCGCTGGATG
TAAAAAAAGGCGACCGGGTCAAGGTGACCATCGTCAACAACTCGCCGGAG
GACGTCCACCCGATGCATTTGCACGGACACTTCTTCCAGGTGCTGAGCAA
AAACGGCCAGCCCGTGTCCGGCTCGCCGCTGGTCAAGGACACCTTGAATG
TGCTGCCAGGCGAGTCCTACGTCGTCGCCTTTGCGGCTGACAATCCCGGC
GAGTGGATGTTCCACTGCCACGACCTGGGGCATGCGGCCAAAGGGATGGT
GTCCGAGGTCAAATACACGGGCTTCCAGCGGGACTTCGTCGTCGATCCGA
CCGTCGGCAACATGCCGGAGTAA.

In another embodiment, the DNA sequence encoding the laccase of the present invention is at least 70% identical to the DNA sequence of SEQ ID NO: 2. In another embodiment, the DNA sequence encoding the laccase of the present invention is at least 75% identical to the DNA sequence of SEQ ID NO: 2. In another embodiment, the DNA sequence encoding the laccase of the present invention is at least 80% identical to the DNA sequence of SEQ ID NO: 2. In another embodiment, the DNA sequence encoding the laccase of the present invention is at least 85% identical to the DNA sequence of SEQ ID NO: 2. In another embodiment, the DNA sequence encoding the laccase of the present invention is at least 90% identical to the DNA sequence of SEQ ID NO: 2. In another embodiment, the DNA sequence encoding the laccase of the present invention is at least 95% identical to the DNA sequence of SEQ ID NO: 2.

In another embodiment, a composition as described herein is an aqueous composition. In another embodiment, a composition as described herein is in the form of a gel.

In another embodiment, a composition as described herein further comprises Cu²⁺. In another embodiment, and particularly with respect to a composition comprising Brevibacillus agri lactase, the composition comprises at least 5 μM, at least 10 μM, at least 15 μM, at least 20 μM, at least 25 μM or at least 30 μM Cu²⁺. In another embodiment the composition comprises 25 μM-35 μM Cu²⁺, preferably about 30 μM Cu²⁺.

In another embodiment, a composition as described herein further comprises xylan. In another embodiment, and particularly with respect to a composition comprising Brevibacillus agri lactase, the composition comprises at least 50 μg/mL, at least 75 μg/mL, at least 80 μg/mL, at least 90 μg/mL, at least 95 μg/mL or at least 100 μg/mL xylan. In yet another embodiment, the composition comprises at most 500 μg/mL, at most 400 μg/mL, at most 300 μg/mL, at most 200 μg/mL, at most 150 μg/mL, at most 140 μg/mL, at most 130 μg/mL, at most 120 μg/mL, at most 110 μg/mL or at most 100 μg/mL xylan. In another embodiment, a composition as described herein further comprises Cu²⁺ and xylan. In another embodiment, a composition as described herein further comprises 30-100 μM Cu²⁺ and 50-150 μg/mL xylan, 45-75 μM Cu²⁺ and 75-125 μg/mL xylan, or about 60 μM Cu²⁺ and about 100 μg/mL xylan. In another embodiment, a composition as described herein further comprises xylan and is devoid of Cu²⁺.

In another embodiment, a composition as described herein further comprises ABTS (2,2′-azino-bis(3-ethylbenzothiazoline-6-sulphonic acid)). In another embodiment, and particularly with respect to a composition comprising Brevibacillus agri lactase, the composition comprises about 0.05% (v/v) to about 0.05% (v/v), about 0.1% (v/v) to about 0.01% (v/v), about 0.01% (v/v) to about 0.05% (v/v) or about 0.01% (v/v) ABTS.

In another embodiment, a composition as described herein further comprises a bivalent metal. It should be appreciated that bivalent metals are known to those skilled in the art. In another embodiment, the bivalent metal is cobalt (Co²⁺). In another embodiment, the bivalent metal is nickel (Ni²⁺). In another embodiment, and particularly with respect to a composition comprising Brevibacillus agri lactase, the composition comprises at least 5 μM, at least 10 μM, at least 15 μM or at least 20 μM bivalent metal.

In another embodiment, a composition as described herein has a pH of 6.5 to 9.5. In another embodiment, a composition as described herein has a pH of 7 to 9. In another embodiment, a composition as described herein has a pH of 7.5 to 8.5. In another embodiment, a composition as described herein has a pH of 7.8 to 8.2.

In another embodiment, a composition as described herein further comprises a surfactant which keeps polyethylene and laccase in contact. In another embodiment, the surfactant is a plastic-binding factor. In another embodiment, the surfactant is a biosurfactant.

In another embodiment, the surfactant is any substance known to those skilled in the art. In another embodiment, the surfactant is a plastic-binding protein. In another embodiment, the surfactant is a glycolipid. In another embodiment, the surfactant is a glycolipid ester such as mannosilerythritol lipid and rhamnolipid; cyclolipbpeptide; cyclopolypeptide; and amphiphatic protein such as surfectin. In another embodiment, the surfactant is a plastic-binding protein such as but not limited to hydrophobin and its homologues.

In another embodiment, the terms “polyethylene” and “plastic” are used interchangeably. In another embodiment, plastic is polyester, polyurethane, polypropylene, polyvinyl chloride, nylon, polystyrene, starch, and any combination thereof. In another embodiment, polystyrene includes poly butylene succinate (PBS), poly butylsuccinate adipate (PBSA), poly lactic acid (PLA), aliphatic polyester, polycaprolactone and any combination thereof. In another embodiment, plastic is biodegradable plastic. In another embodiment, biodegradable plastic is plastic that keeps its function during a use state and will be degraded to a simpler molecular level by the function of the composition of the invention. In another embodiment, plastic to be degraded may take any form such as emulsion and solid pellet depending the type of degradation reaction. In another embodiment, polyethylene is treated by thermo-oxidation. In another embodiment, polyethylene is treated by oxidation. In another embodiment, polyethylene is treated by both thermo-oxidation and oxidation.

In another embodiment, the composition comprises a biologically pure culture of B. borstelensis and plastic. In another embodiment, the composition comprises a biologically pure culture of B. agri and plastic. In another embodiment, the composition comprises a mixture of biologically pure cultures of Brevibacillus agri, Brevibacillus borstelensis, and plastic. In another embodiment, the composition comprises a mutant derived Brevibacillus agri or B. borstelensis which retains the plastic biodegrading activity thereof at a temperature from about 60° C. to about 100° C.

Determination of an effective, biodegrading amount of microorganism as described in the claimed invention is within the knowledge of one skilled in the art. Various methods exist in which one can determine the amounts of the bacteria required to effectively degrade the waste of interest.

### Method

In another embodiment, polyethylene biodegradation through laccase comprises two steps: firstly enzyme adheres to the polyethylene substrate and then catalyzes a hydrolic cleavage. In another embodiment, laccase disintegrates polyethylene into short chains of oligomers, dimers, and monomers. In another embodiment, laccase disintegrates polyethylene into short chains of oligomers, dimers, and monomers that act as the sole source of carbon and energy to a bacterium of the invention. In another embodiment, during biodegradation the monomers are further mineralized. In another embodiment, the biodegradation process of the invention is a depolymerisation process.

In another embodiment, the biodegradation process of the invention results in the end products: carbon dioxide (CO₂), water (H₂O) and/or methane (CH₄). In another embodiment, the biodegradation process begins with a microorganism having the ability to enzymatically degrade polyethylene. In another embodiment, the biodegradation process begins with the purified enzyme. In another embodiment, the process includes adherence of laccase to the plastic surface. In another embodiment, the laccase cleavages the plastic's polymer chains resulting in erosion of the plastic surface i.e biodegradation and the end products CO₂, H₂O and CH₄ are produced. In another embodiment, laccase oxidizes the hydro-carbon backbone of polyethylene. In another embodiment, the process as described herein is eco-friendly.

### Enzymatic

In another embodiment, the invention provides a method for decomposing polyethylene, comprising the step of contacting polyethylene with a laccase. In another embodiment, the invention provides a method for decomposing polyethylene, comprising the step of contacting polyethylene with a laccase having an optimal specific activity at a temperature range of 60° C. to 100° C. In another embodiment, the method further includes contacting laccase with Cu²⁺.

In another embodiment, the method further includes maintaining polyethylene and laccase at a temperature range of 50° C. to 100° C. In another embodiment, the method further includes maintaining polyethylene and laccase at a temperature range 60° C. to 100° C. In another embodiment, the method further includes maintaining polyethylene and laccase at a temperature range of 70° C. to 90° C. In another embodiment, the method further includes maintaining polyethylene and laccase at a temperature range of 75° C. to 85° C. In another embodiment, the method further includes maintaining polyethylene and laccase at a temperature range of 77° C. to 83° C.

In another embodiment, the method further includes maintaining polyethylene and laccase at a pH of 7 to 10. In another embodiment, the method further includes maintaining polyethylene and laccase at a pH of 7 to 9. In another embodiment, the method further includes maintaining polyethylene and laccase at a pH of 7.5 to 8.5. In another embodiment, the method further includes maintaining polyethylene and laccase at a pH of 7.8 to 8.2.

In another embodiment, the method of the invention further includes incubation of said laccase with said polyethylene prior to the biodegradation reaction. As would be appreciate to a skilled artisan, the incubation period may depend on the specific lacaase used in the reaction. In another embodiment, said incubation is at least 1 day, at least 2 days, at least 3 day, at least 4 days, at least 5 day, at least 6 days, at least 7 day or at least 14 days.

### Bacterial

In another embodiment, the invention provides a method for decomposing polyethylene, comprising the step of contacting polyethylene with Brevibacillus borstelensis, B. agri, or a combination of Brevibacillus borstelensis and Brevibacillus Agri, wherein polyethylene is the only carbon source for the bacteria. In another embodiment, the method further includes contacting the bacteria with Cu²⁺. In another embodiment, the method further includes contacting the bacteria with xylan. In another embodiment, the method further includes contacting the bacteria with Cu²⁺ and xylan.

In another embodiment, the invention provides a method for decomposing polyethylene, comprising the step of contacting polyethylene with bacteria expressing the laccase of the invention. In another embodiment, bacteria consists a bacterial strain. In another embodiment, bacteria consists an isolated bacterial strain.

In another embodiment, the bacterial reaction of the method of the invention is performed at a temperature range of 30° C. to 55° C. In another embodiment, the bacterial reaction of the method of the invention is performed at a temperature of 35° C. to 50° C. In another embodiment, the bacterial reaction of the method of the invention is performed at a temperature of 35° C. to 45° C. In another embodiment, the bacterial reaction of the method of the invention is performed at a temperature of 37° C. to 43° C.

In another embodiment, the bacterial reaction of the method of the invention is performed at a pH of 7 to 9.5. In another embodiment, the bacterial reaction of the method of the invention is performed at a pH of 7 to 9. In another embodiment, the bacterial reaction of the method of the invention is performed at a pH of 7.5 to 8.5. In another embodiment, the bacterial reaction of the method of the invention is performed at a pH of 7.8 to 8.8.

### Biodegradation Reaction

In another embodiment, the biodegradation reaction according to the present invention is performed in any reaction system (e.g., aqueous solution or solid system) and any conditions known to those skilled in the art depending on its purpose and scale.

In another embodiment, the biodegradation reaction includes a strengthened hydrophobic interaction between the surfactant and the plastic in order to effectively attach to the plastic and to obtain an advantage of the present invention. In another embodiment, the present methods comprise a step of mixing the surfactant and the plastic in a low water activity condition, and a step of biodegrading the plastic with the use of the plastic-biodegrading enzyme/bacteria in a high water activity condition.

In another embodiment, the phrase “water activity” is a factor well known to those skilled in the art, and is defined as a ratio between the vapor pressure of a solution comprising a solute and that of pure one. In another embodiment, “low water activity condition” is a condition wherein the surfactant can significantly attach to a hydrophobic surface of the plastic.

In another embodiment, a reaction includes mixing the surfactant and the plastic in film or pellets in the low water activity condition so that an effective amount of the surfactant attaches to the plastic, and increase the water activity to promote the biodegradation of the plastic with the plastic-biodegrading enzyme/bacteria.

In another embodiment, the reaction is performed in a low salt concentration (less than 4%) condition.

This invention is illustrated in the Experimental Details section which follows. These sections are set forth to aid in an understanding of the invention but are not intended to, and should not be construed to, limit in any way the invention as set forth in the claims which follow thereafter.

As used herein, the singular forms “a”, “an”, and “the” include plural forms unless the context clearly dictates otherwise. Thus, for example, reference to “a therapeutic agent” includes reference to more than one therapeutic agent.

Unless specifically stated or obvious from context, as used herein, the term “or” is understood to be inclusive.

The term “including” is used herein to mean, and is used interchangeably with, the phrase “including but not limited to.”

As used herein, the terms “comprises,” “comprising,” “containing,” “having” and the like can have the meaning ascribed to them in U.S. patent law and can mean “includes,” “including,” and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In another embodiment, the term “comprise” includes the term “consist”.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

### Example 1

### Biodegradation of Polyethylene by Newly Isolated Laccase from Soil Bacteria

This study focused on induction and optimization of the activity of bacterial laccase which was found to be involved in plastic biodegradation.

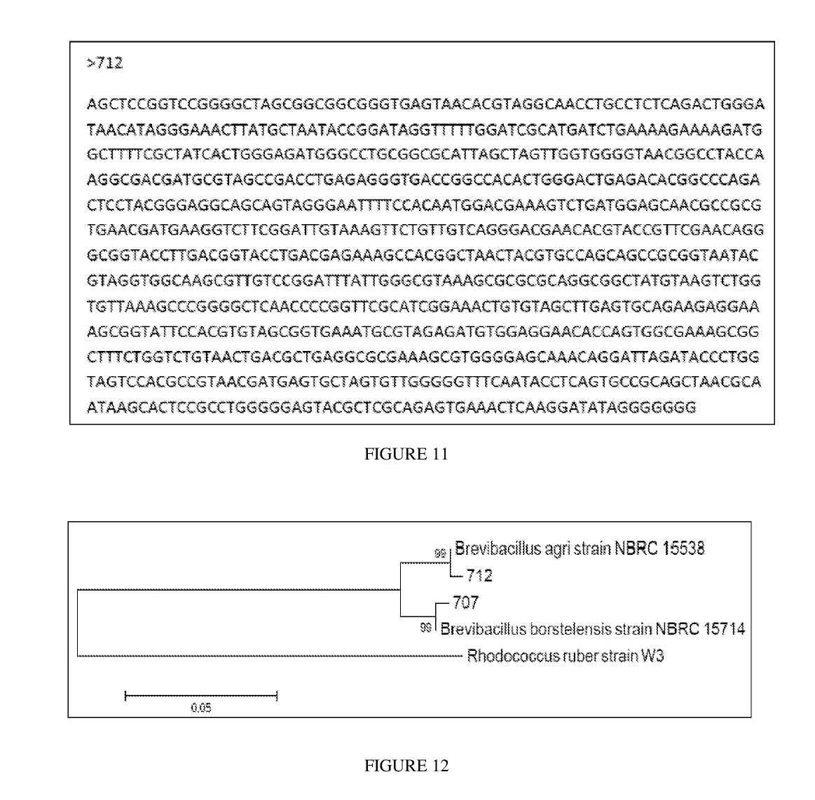

It was found that two soil bacteria showed favorable polyethylene biodegradation ability. Both bacteria were sequenced according to their 16SrRNA gene and were identified as Brevibacillus borstelensis and Brevibacillus agri. The 16SrRNA gene is shown in FIG. 11. FIG. 12 shows a phylogenetic tree of both strains based on their 16SrRNA sequence with rhodococcus ruber as an external strain.

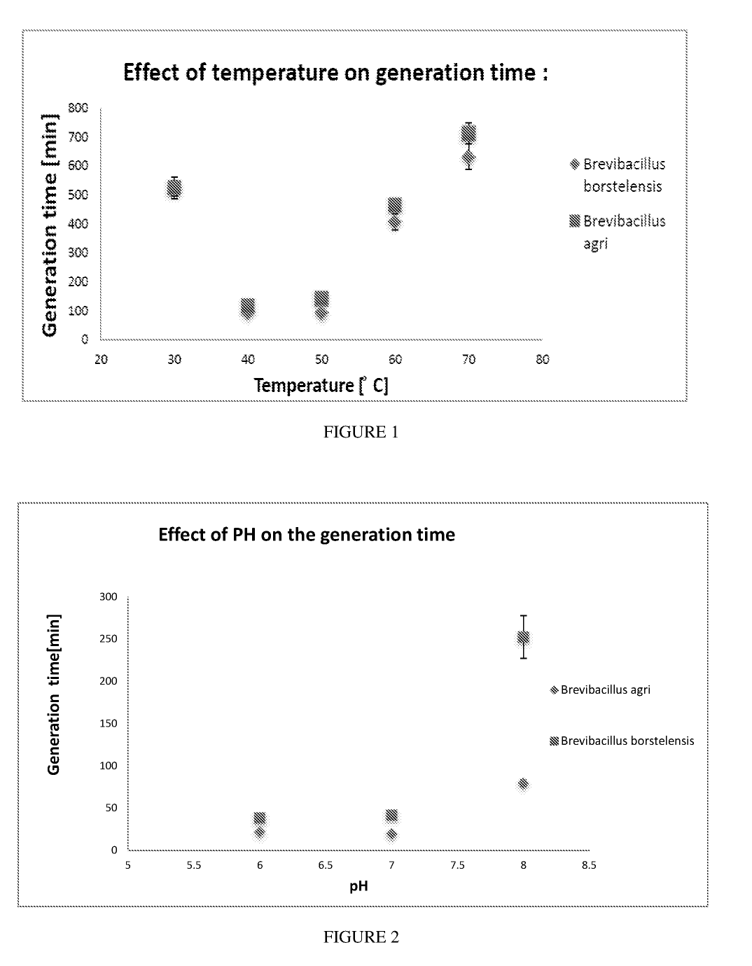

Characterization of the two strains revealed that they are thermophilic, with optimal growth temperature of 40° C. for Brevibacillus borstelensis and 45° C. for Brevibacillus agri (FIG. 1). It was also found that the optimal pH growth condition are between pH=6.5 to pH=7 (FIG. 2).

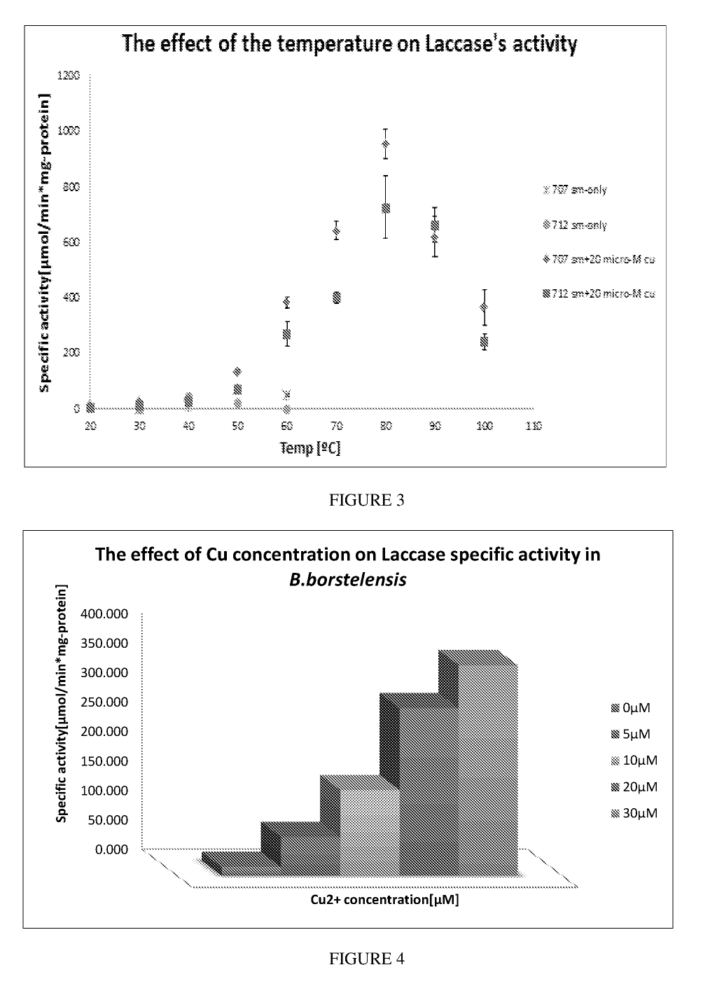

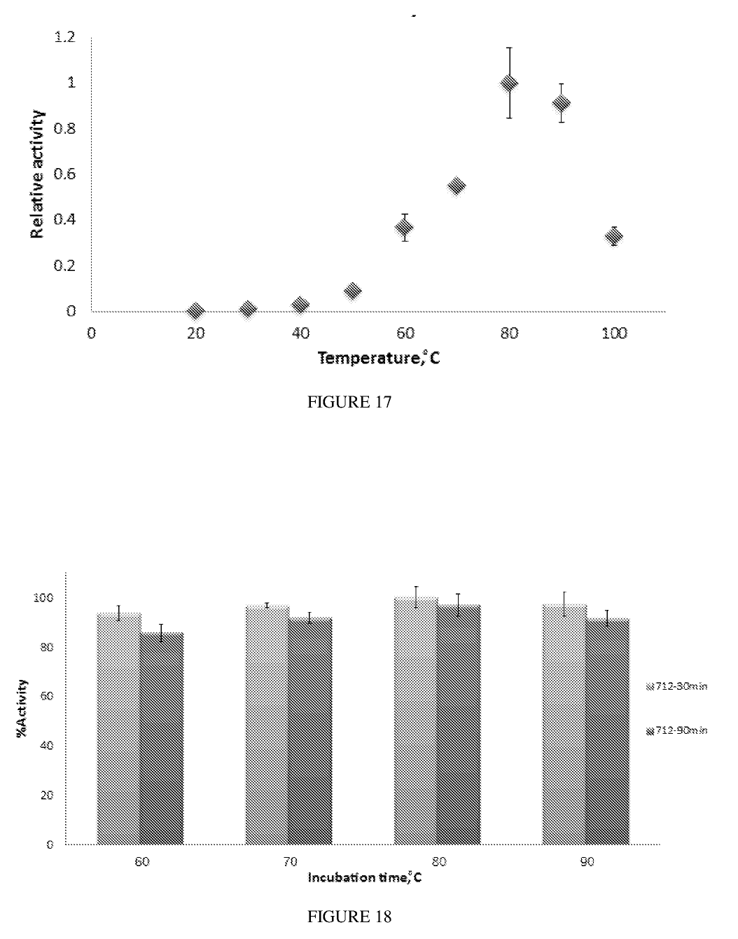

Further evidence showed that both strains produce extra-cellular laccase enzymes that exhibited an optimal activity at 80° C. (FIG. 3 and FIG. 17). The optimal temperature is relatively high compared to the optimal growth temperature of the bacterium and it is possible that it's derived from an evolutionary remainder from a hyper thermophile ancestor of the bacterium that used the enzyme.

In order to know the effect of incubation at high temperatures on the enzyme, the survival of laccase enzyme in different temperatures was examined (FIG. 18). During this test the laccase was incubated in the different temperatures for 30 or 90 minutes and then an activity test was made. It was found that the temperature in which the enzyme survived best in Brevibacillus agri was 80° C., in this temperature the enzyme showed a decrease of 4% in its activity.

When the bacteria were incubated with polyethylene, it was noticed that B. agri had a better biodegradation ability compared to B. borstelensis (FIG. 8).

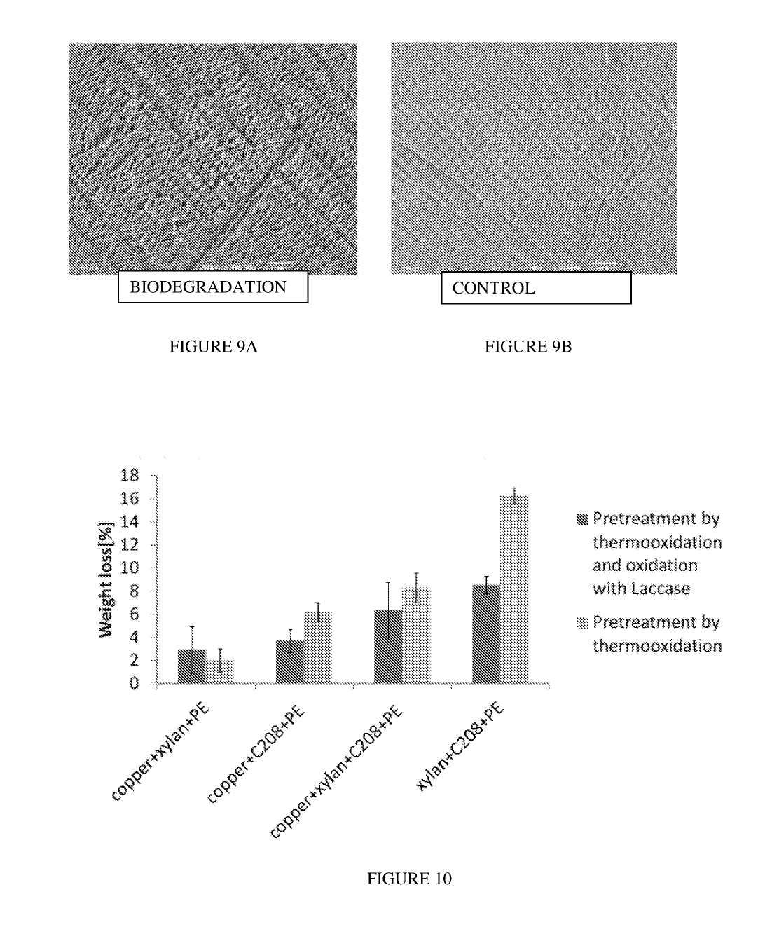

Biodegradation of polyethylene was clearly visible to the eye when magnified 10,000 times using SEM microscopy compared with the control (FIG. 9).

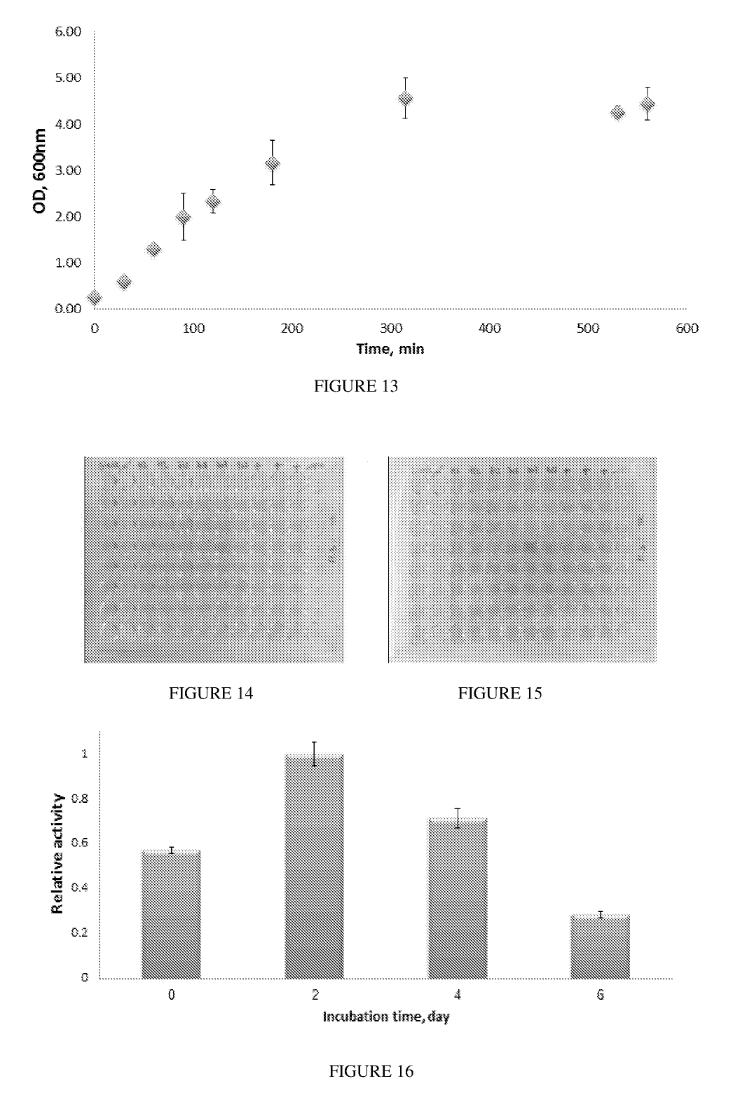

The bacterial growth of Brevibacillus agri was characterized. As shown in FIG. 13 the bacteria entered the stationary phase after 300 minutes. Further, the optimal growth time for activity analysis was made, it was found that the optimal analysis time for Brevibacillus agri was two days (FIG. 16).

### Example 2

### Extracellular Laccase Excretion

The excretion of extracellular laccase was examined in the presence of 2,6-DiMethoxyPhenol (DMP), found to be involved in the biodegradation process. The oxidation of this substrate by the enzyme leads to a change in the solution color to a yellowish-brownish color. After 19 hours of incubation of the extracellular enzyme with 30 mM DMP in room temperature, a brownish color was received (FIG. 15), when compared to the blank (FIG. 14). This indicated an excretion of the laccase enzyme to the extracellular medium.

### Example 3

### The Effect of Different Additives on Laccase Activity

In order to improve the enzyme activity, the effect of different additives addition to the bacterial growth medium was examined. The effect on both laccase induction and activity were examined. The following additives were examined: Copper (Cu²⁺), ABTS, Xylan, copper and Xylan synergism, Cobalt and Nickel.

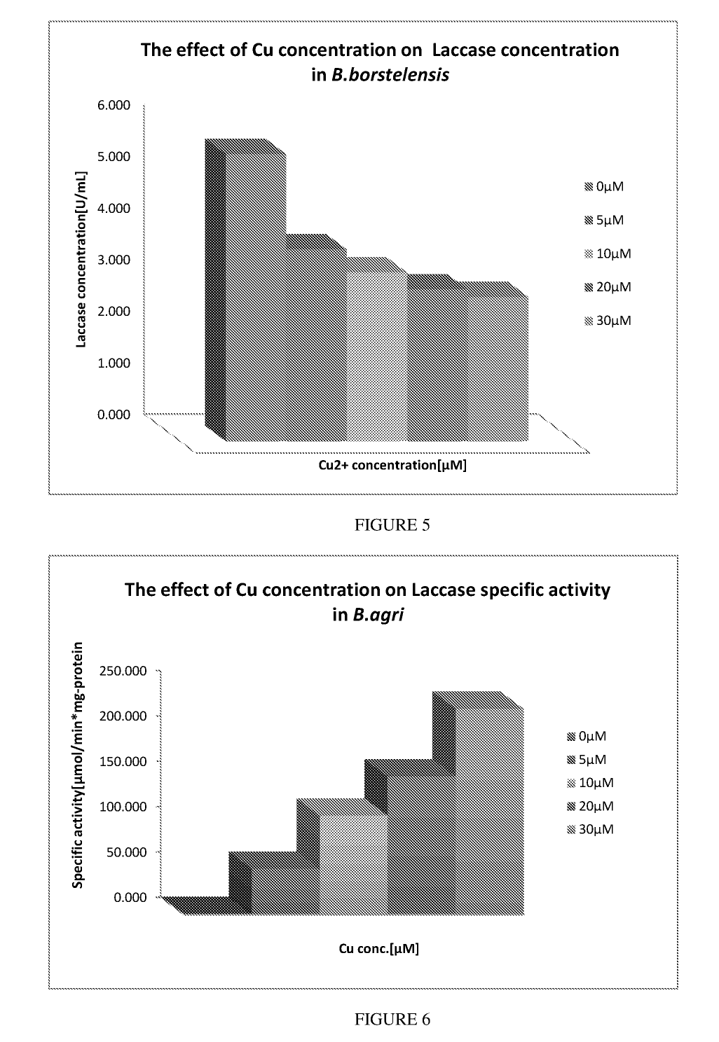

The effect of Cu²⁺ addition to the growth media on induction and activity of Laccase in both bacteria was evaluated. In B. borstelensis, it was found that the addition of growing concentrations of Cu2+ lead to lower induction of Laccase, but with higher specific activity (FIGS. 4 and 5).

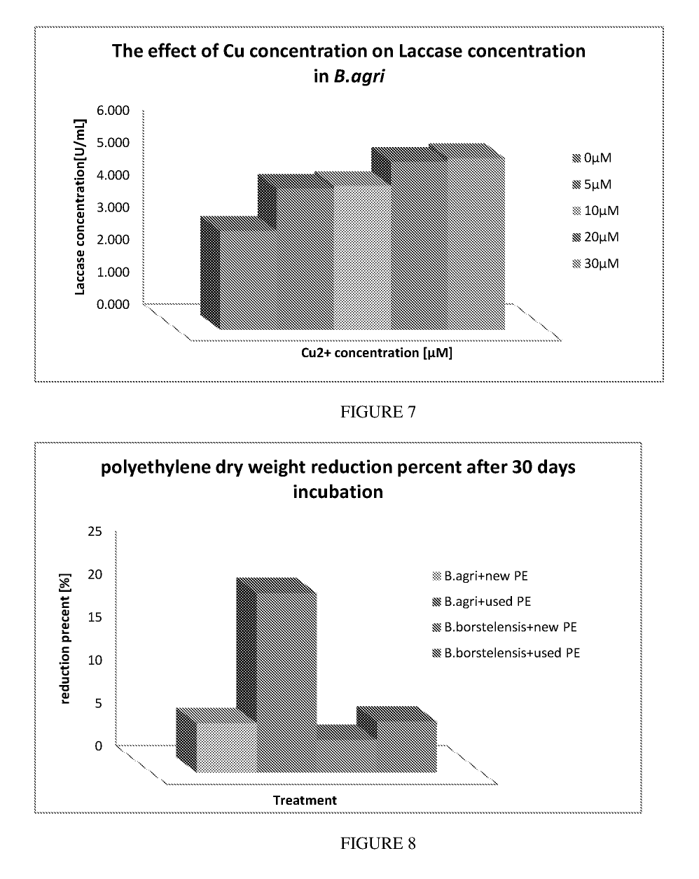

In B. agri, it was found that the addition of growing concentrations of Cu2+ lead to improved induction and specific activity of laccase (FIG. 6). Further, the effect of Cu concentration on laccase concentration in B. agri was monitored (FIG. 7). The positive effect of copper on the enzyme induction and activity can be explained by the fact that copper constitutes part of the enzyme catalytic site. Therefore increasing levels of copper will lead to the production of more active enzymes.

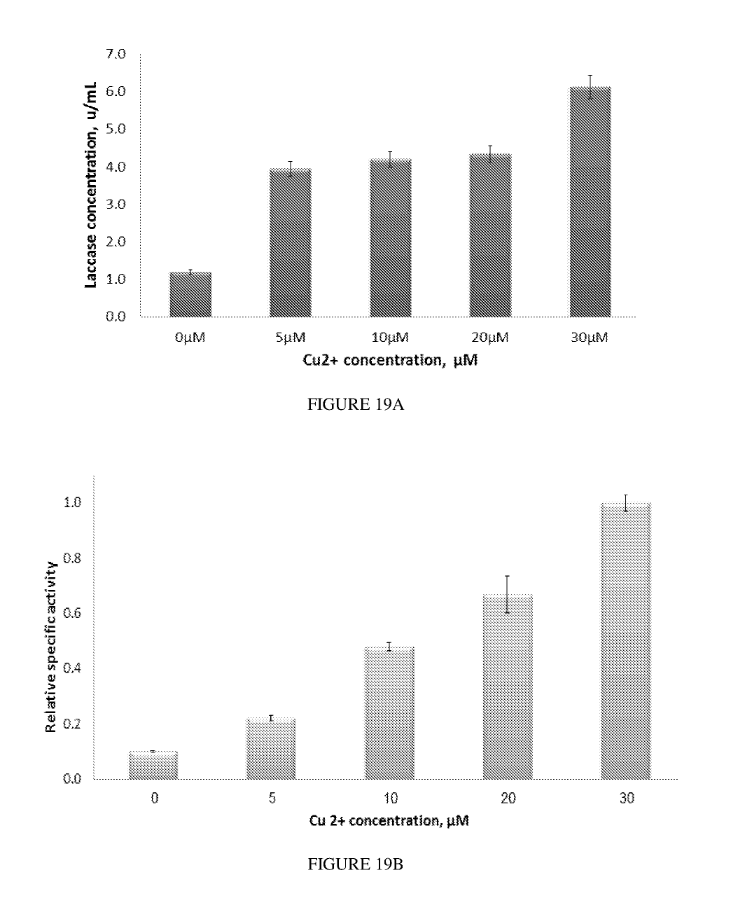

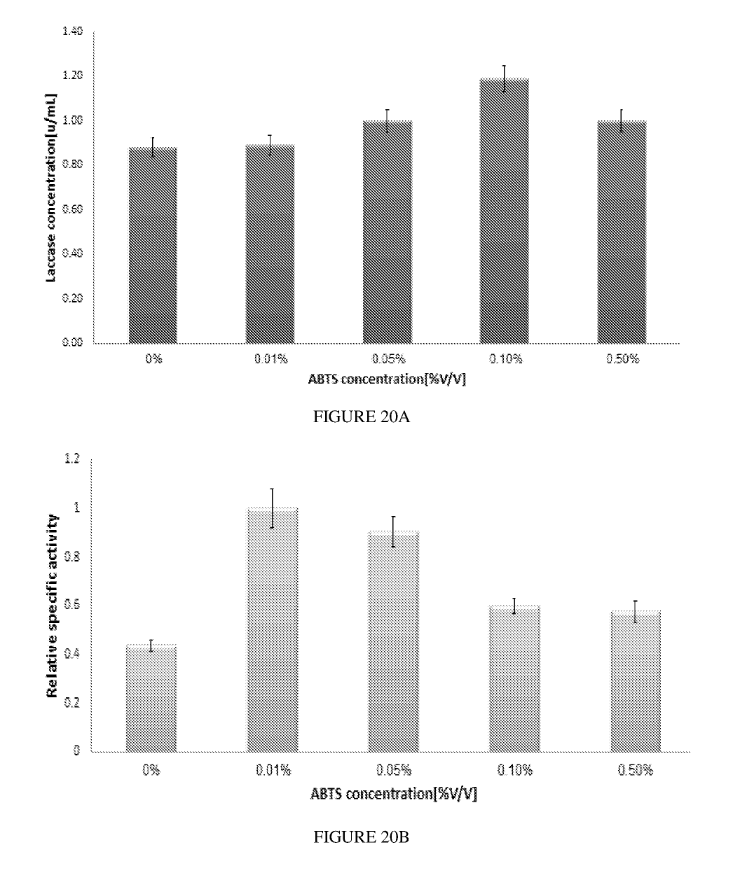

ABTS also known as 2,2′-azino-bis(3-ethylbenzothiazoline-6-sulphonic acid) is a known substrate and laccase mediator. When analyzing the effect of ABTS it was assumed that its addition to the reaction medium will improved the enzyme activity. ABTS concentrations of 0.1% (v/v) led to optimal laccase induction in Brevibacillus agri (FIG. 19). ABTS concentrations of 0.01% (v/v) led to optimal laccase specific activity in Brevibacillus agri (FIG. 20).

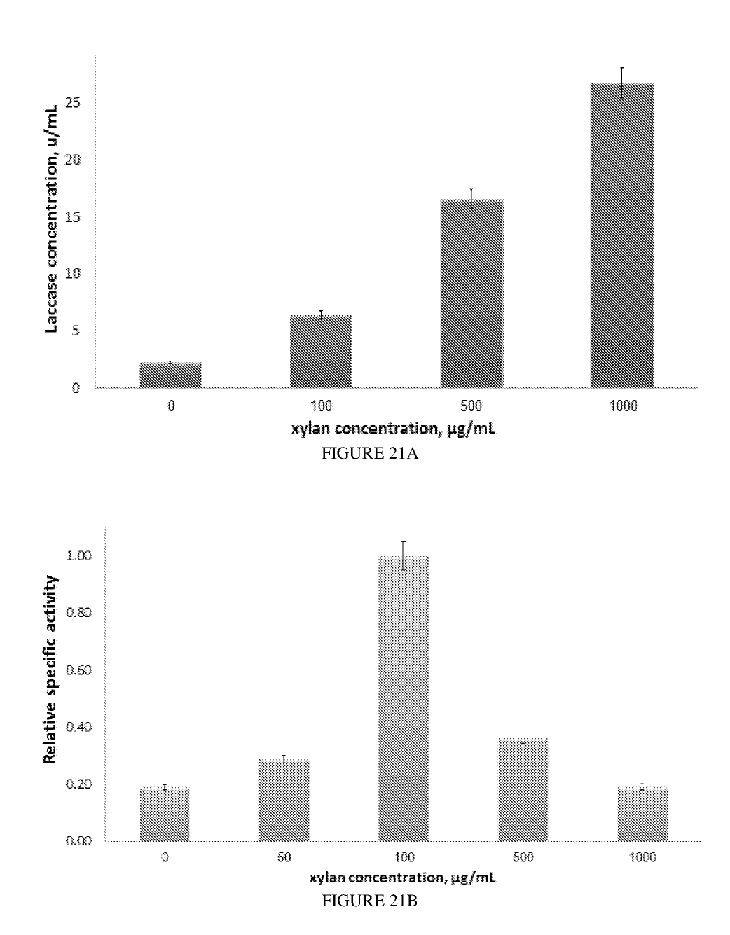

When examining the effect of xylan, it was found that growing xylan concentrations lead to an increase in laccase induction in Brevibacillus agri (FIG. 21A). Xylan concentration of 100 μg/mL leads to optimal laccase specific activity (FIG. 21B).

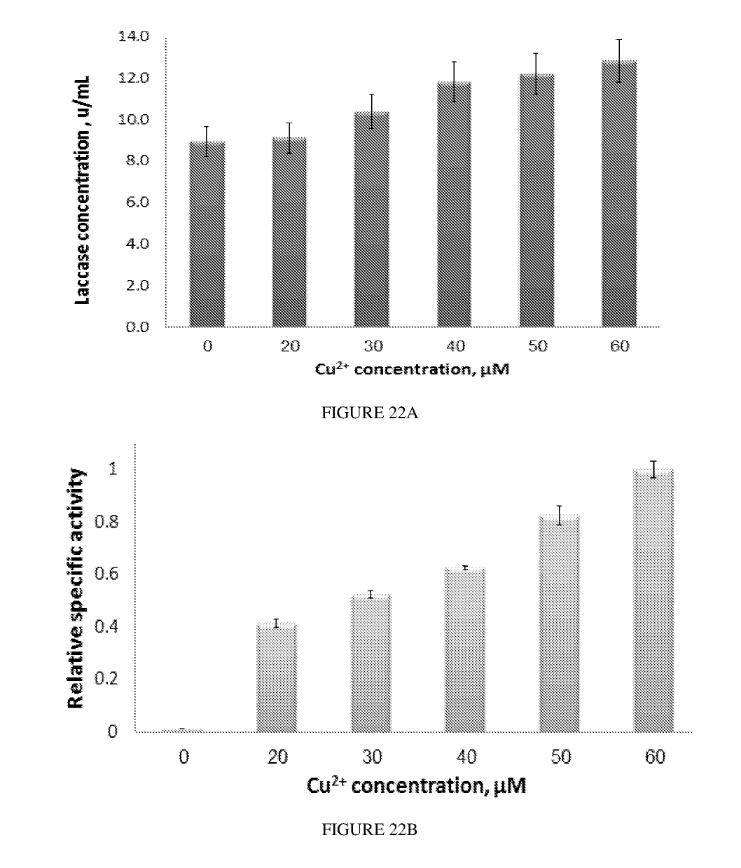

When analyzing the effect of xylan and copper synergism, it was found that growing copper concentrations led to an increase in laccase both induction and specific activity in Brevibacillus agri (FIGS. 22A-B). It should be mentioned that when comparing these results to the ones that were received for copper addition—the induction level is indeed higher in copper and xylan induction.

The effect of bivalent metals on laccase induction and activity was also examined. The chosen metal were cobalt, found to be capable of replacing the copper in the catalytic site, and nickel, a known bivalent metal with oxidizing abilities.

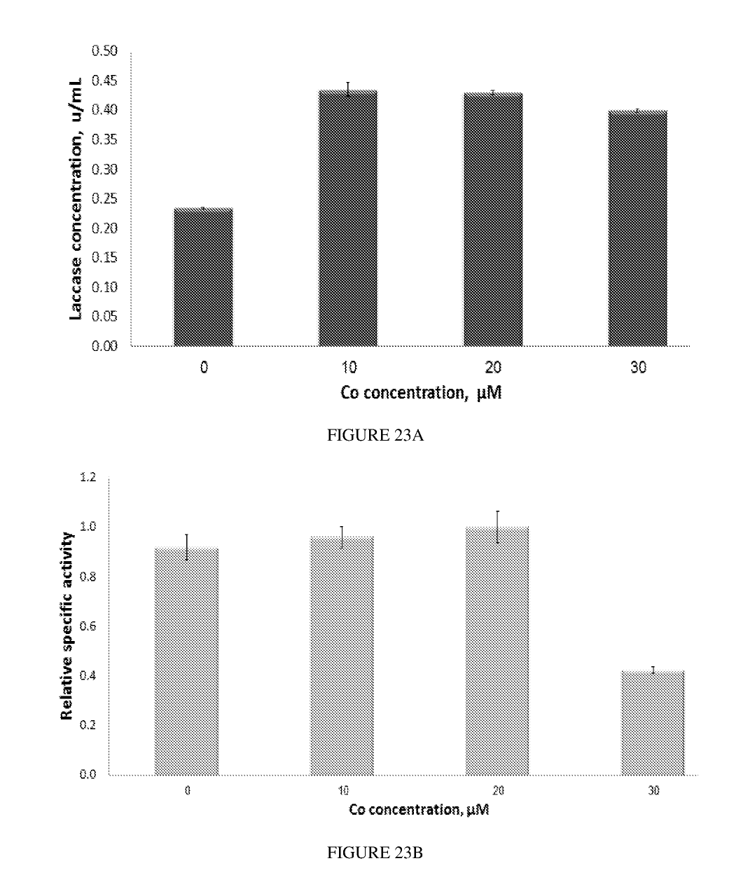

When examining the effect of cobalt concentration on laccase induction and activity, it was found that cobalt concentration of 20 μM led to optimal laccase induction (FIG. 23A) and a concentration of 10 μM led to optimal laccase specific activity in Brevibacillus agri (FIG. 23B).

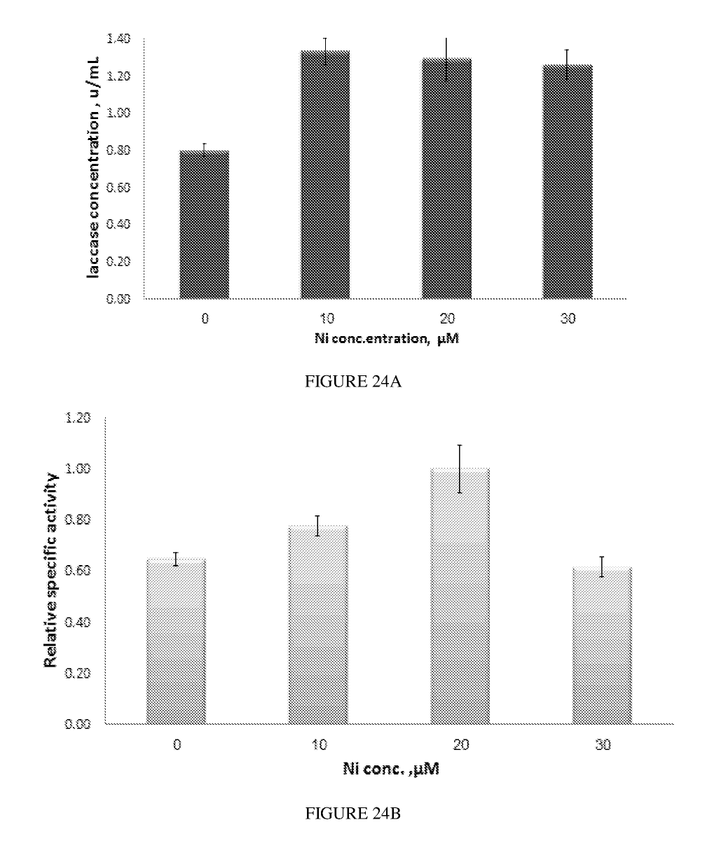

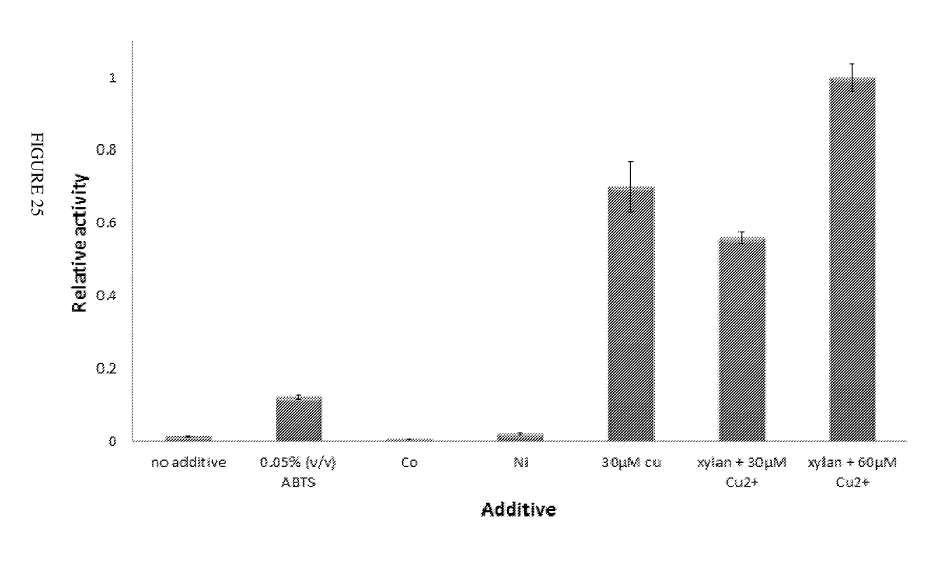

When analyzing the effect of nickel concentration on laccase induction and activity, it was found that nickel concentration of 20 μM led to optimal laccase induction (FIG. 24A) and nickel concentration of 10 μM led to optimal laccase specific activity in Brevibacillus agri (FIG. 24B).

When comparing the effect of the different additives on laccase activity (FIG. 25), it was found that the optimal additives are 30 μM Cu2+ or 100 μg/mL xylan+60 μM Cu2+.

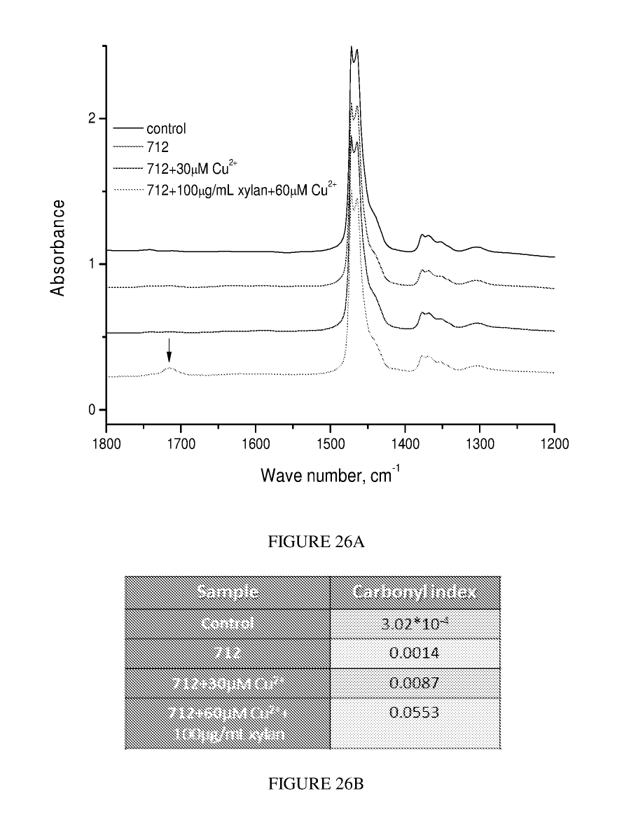

Further examined was the effect of the different additives, copper and copper and xylan synergism, and 7 days incubation with laccase prior to a biodegradation with polyethylene (PE). According to the (fourier transform infrared spectroscopy) FTIR analysis (FIGS. 26A-B), when the PE wasn't incubated with the laccase prior to the biodegradation experiment, it was seen that biodegradation experiment in the presence of 100 μg/mL Xylan and 60 μM copper yielded the highest carbonyl index which indicates that a better biodegradation process took place.

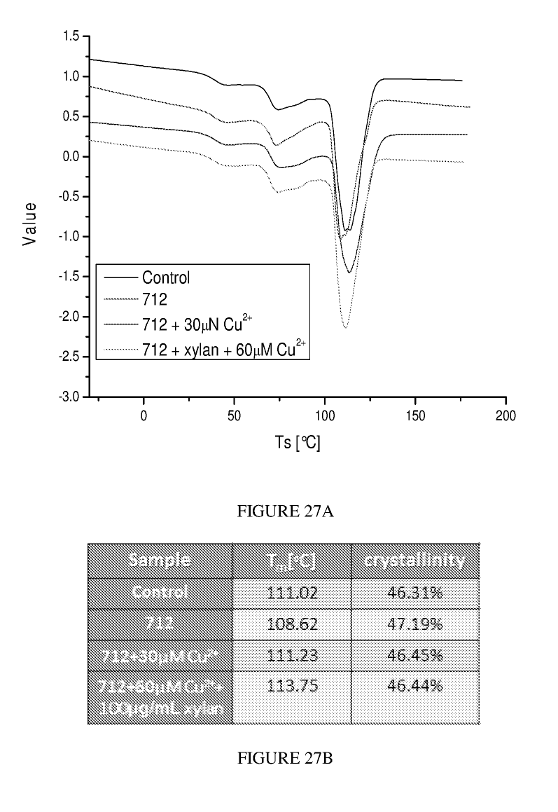

According to (differential scanning calorimeter) DSC analysis (FIGS. 27A-B), no significant difference between the samples crystallinity was received. It may be assumed that at the beginning of the biodegradation experiment, the biodegradation of PE starts from amorphous area in the PE, and the crystallization percent of the PE increases. Then the biodegradation process continues in the crystalline area of the polymer, steric disturbances in the crystalline area of the PE occur as a cause of the oxidation of this area, and the crystallization percent decrease.

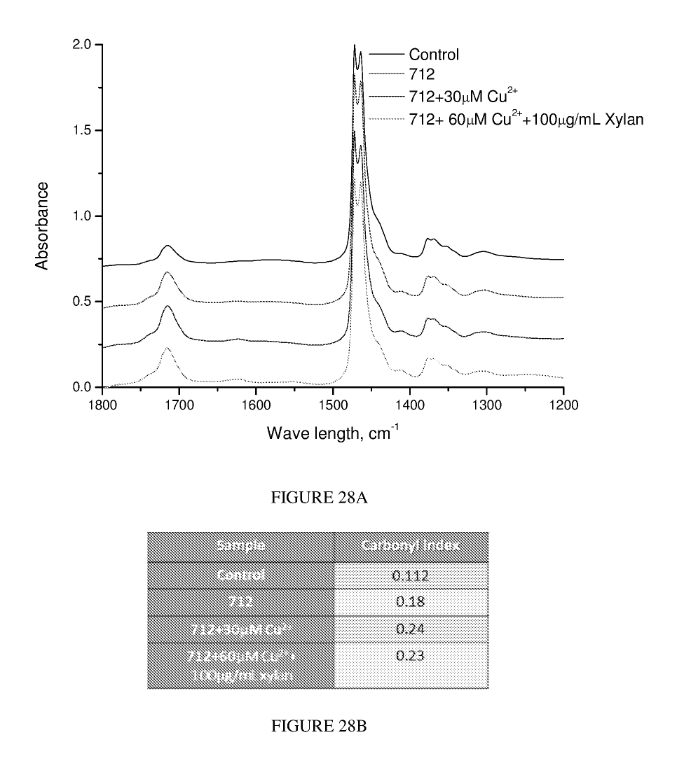

When the PE was incubated with the laccase prior to the biodegradation experiment, higher carbonyl index values in FTIR analysis were received for all samples (FIG. 28A). Here, a higher carbonyl index was received in all samples compared to the control (FIG. 28B), though there's not a significant difference between the different treatments, especially when comparing the effect of copper addition to the copper and xylan addition to the bacterial growth culture.

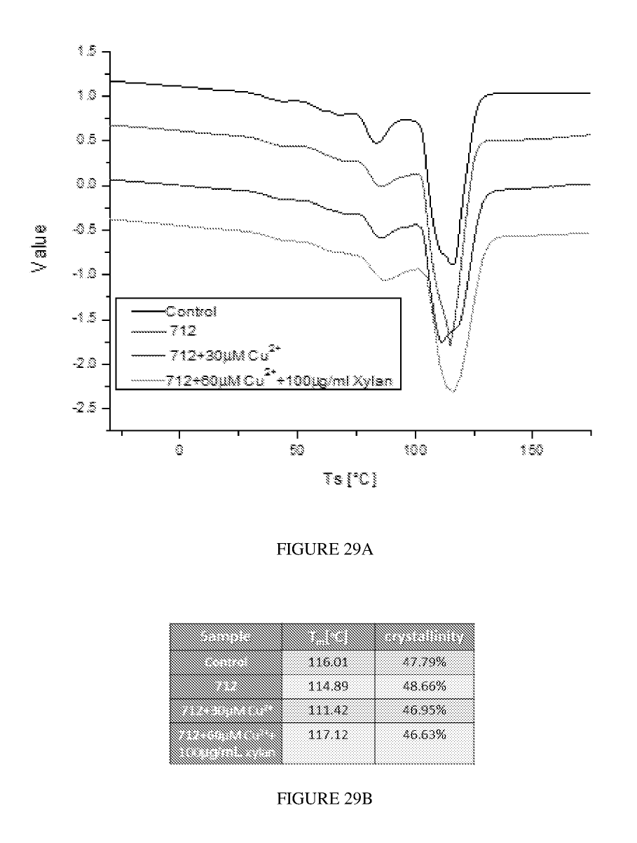

When the PE was incubated with the laccase prior to the biodegradation experiment, higher crystallinity and melting temperatures of the polymer were also received (FIG. 29), which might indicate that the amorphous part of the polymer was broken and a more stable polymer was received. In both of the experiments, the crystallinity level of the sample that was incubated with 712 strain was higher than in the other experiments, it might be connected to the theory that the biodegradation starts from the amorphous part of the polymer the crystallinity increases and then in some point the biodegradation of the crystalline area start and the crystallinity decreases.

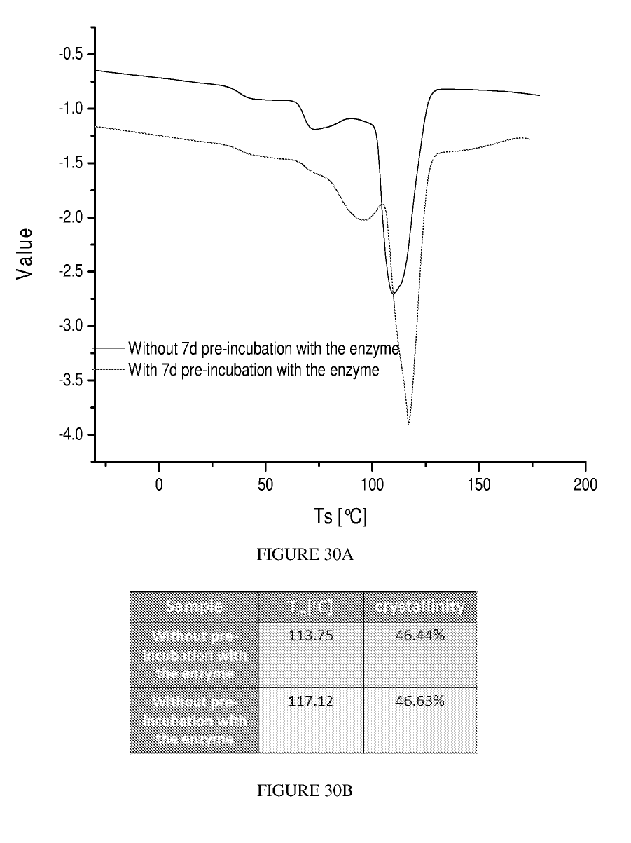

When analyzing the effect of incubation with the enzyme prior to the biodegradation experiment, it is clear that the incubation led to formation of PE with different thermal properties such as melting temperature although there isn't a considerable difference in the polymer crystallinity. This change in the melting temperature indicates that a more stable polymer is obtained (FIG. 30)

### Example 4

### Biodegradation of Polyethylene by Laccase of Rhodococcus Ruber Bacteria after 7 days Incubation with Xylan and/or Copper

Similarly to the previous example this study focused on induction and optimization of the activity of bacterial laccase which was found to be involved in plastic biodegradation.

In this study, it was found that Rhodococcus ruber (C208) bacteria showed favorable polyethylene biodegradation ability.

Initial optimization of C208 polyethylene bidegradation activity revealed that xylan positively impacts this C208, laccase, polyethylene bidegradation activity. Specifically, the results in FIG. 10 show that xylan but not copper induces the C208 laccase activity.

### Example 5

### The Effect of Incubation Time on PE Oxidation by Laccase Enzyme

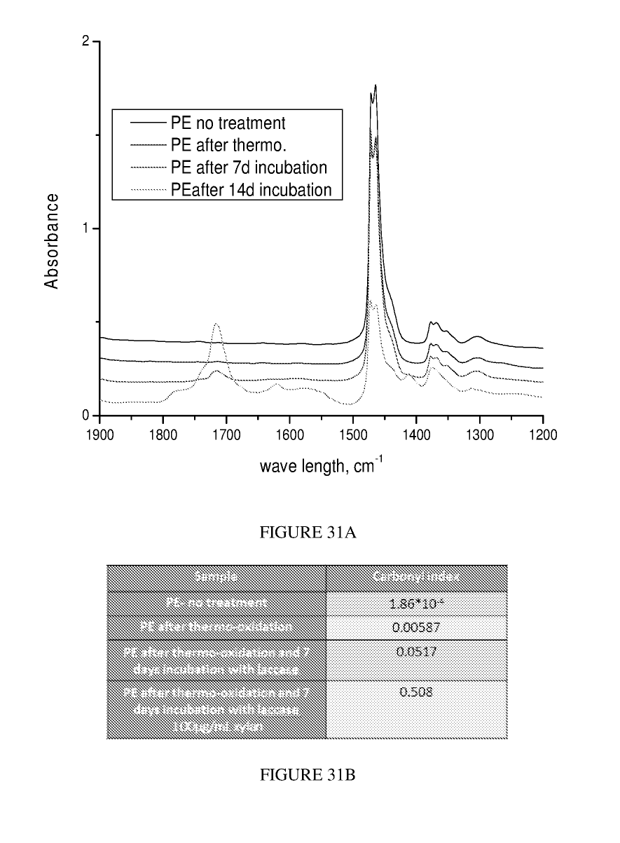

When analyzing the effect of the incubation time of the PE with Laccase using FTIR analysis, it is clear that as the incubation time of the PE with laccase was longer, a higher carbonyl index values were received. Here, the highest carbonyl index was received when the PE sample was incubated with laccase for 14 days (FIG. 31).

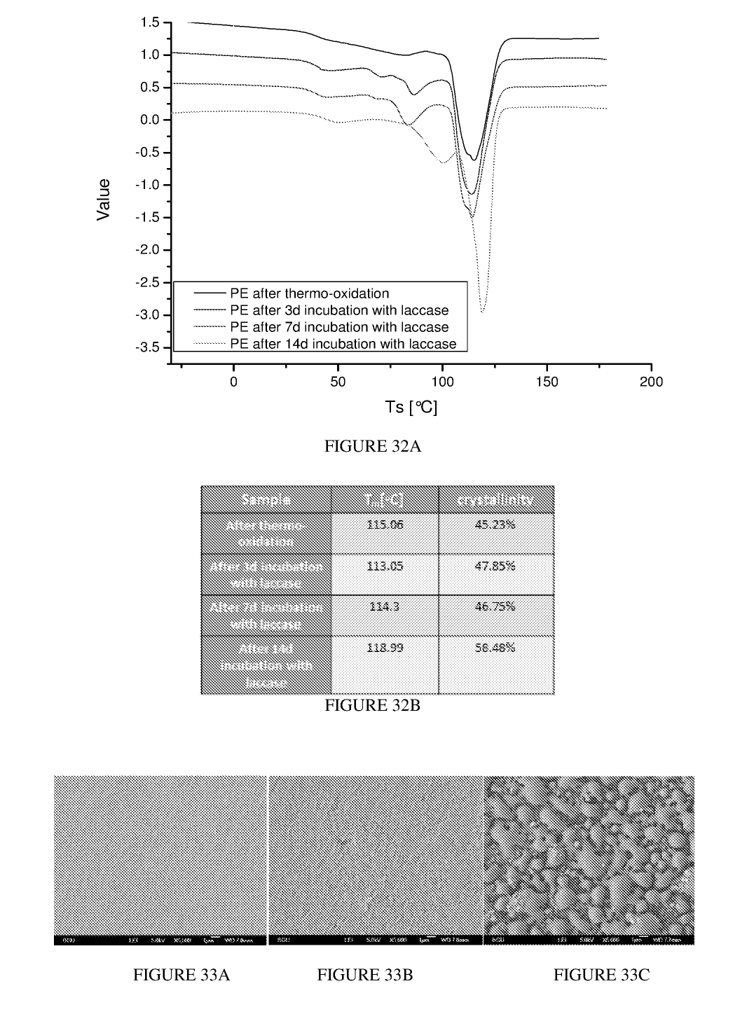

When analyzing the same results using DSC analysis, the results receives were consistent with the FTIR results. In the DSC results it is clear that the PE samples went through a change when compared to the original samples. The melting temperatures changed as well as the crystallinity in the 14 days incubation sample (FIG. 32).

The samples of the previous results were analyzed using scanning electron microscopy (SEM) in order to see if there was any change in the surface of the PE. It can be seen in FIG. 33 that when comparing the PE after 14 days incubation with laccase (FIG. 33C) to the control (FIG. 33B) and to the original PE (FIG. 33A) a considerable change in the PE surface was received. While the surface of the control and the original untreated PE looks quite smooth, after incubation with laccase it looks inflated, or even broken. A possible explanation for this phenomenon is that the laccase enzyme oxidized the surface of the PE and it is supported by the FTIR analysis discussed above.

### Example 6

### Expression of the Enzyme from 712 Bacterial Strain in E. Coli

Based on NCBI website, multicopper oxidase gene (ACCESSION ELK42526) in Brevibacillus agri strain was sequenced. The primers that were used in this process are: Lac-agri F, Lac-agri R, Lac-F3′, Lac-F5′.

| (SEQ ID NO: 4) | |
|---|---|
| Lac-agri F: | 5′ ATGAACAAATCATCGTTACGAAG 3′ |
| (SEQ ID NO: 5) | |
| Lac-agri R: | 5′ TTACTCCGGCATGTTGCCGACGG 3′ |
| (SEQ ID NO: 6) | |
| Lac-F3′: | 5′ TCATTTTCGCGTCGCTCATGTTCG 3′ |
| (SEQ ID NO: 7) | |
| Lac-F5′: | 5′ AATGTGCTGCCAGGCGAGTCCTAC 3′ |

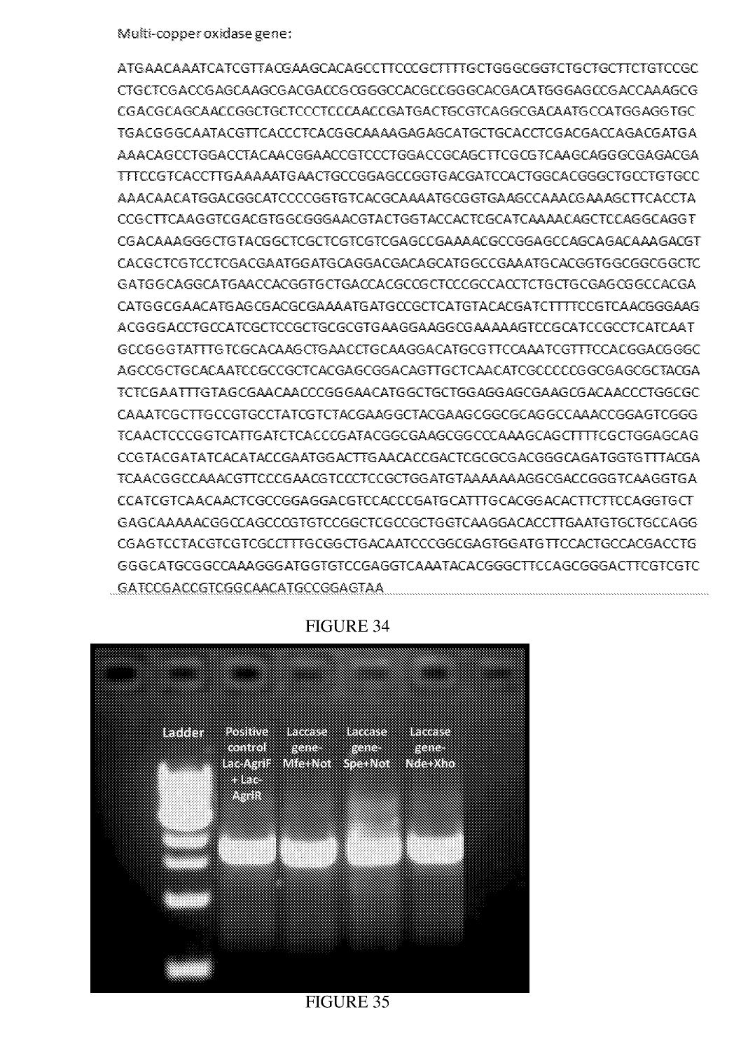

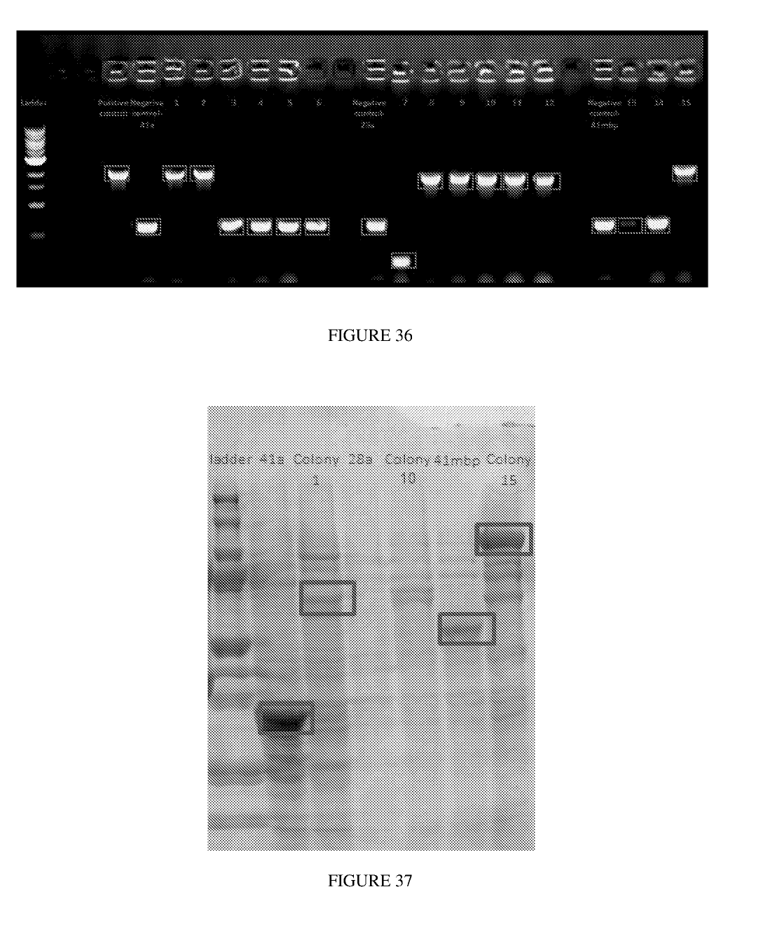

The sequence received is sown in FIG. 34. Based on this sequence, primers for pET41a, pET28, and pET41-mbp were designed. The Primers were designed using the Geneious program and the NEB cutter website.

To cut pET41a and pET41-mbp, the following primers were used:
Fr-Lac-MfeI-pET41(MfeI restriction site):

(SEQ ID NO: 8)
5′-ATGCTACAATTGATATGAACAAATCATCGTTACGAAGC-3′

Fr-Lac-SpeI-pEt41 (Spel restriction site):

(SEQ ID NO: 9)
5′-TAGCTAACTAGTATGAACAAATCATCGTTACGAAGC-3′

Rev-Lac-NotI-pET41 (NotI restriction site):

(SEQ ID NO: 10)
5′-ATGTCATGCGGCCGCCTCCGGCATGTTGCCGACGGTCG-3′

To cut pET28a,the following primers were used:
Fr-Lac-Nde-pET28 (Nde restriction site):

(SEQ ID NO: 11)
5′-ATCGTACCATATGAACAAATCATCGTTACGAAGC-3′

Rev-Lac-Xho-pET28 (Xho restriction site):

(SEQ ID NO: 12)
5′-ATGCTACCTCGAGTTTACTCCGGCATGTTGCCGACGGTCG-3′

The amplification\PCR products of the multi-copper oxidase gene with the primers mentioned above were analyzed using an 1% agarose gel (FIG. 35). The gel clearly indicated a good amplification of the gene using the primers.

After being cut and ligated, the plasmids were transformed into compotent E. Coli bacteria Clooni strain, and a colony-PCR was performed. The analysis of the PCR products using 1% agarose gel (FIG. 26) indicated that colonies 1, 2, 8, 9, 10, 11, 12 and colony 15 are suspected to be positive to the gene when compared to the positive control (gene amplified from the Brevibacillus agri). A laccase enzyme gene amplified from the original bacteria (Brevibacillus agri) served as a positive control. For negative control, an amplification of the unmodified plasmids used, using the same plasmids that were used in analyzing the modified plasmid.

The plasmids of all positive colonies (colonies number 1, 2, 8, 9, 10, 11, 12, 15) were cleaned and sequenced and colonies number 1, 2, 10 and 15 were true positive according to an alignment made in Geneious program (not shown). The positive plasmids were transformed into Competent E. Coli bacteria (BL21 strain) and the enzyme expression by those bacteria was analyzed on a 10% SDS electrophoresis page (FIG. 37). Plasmids 41a, 28a and 41 mbp were used as negative control and were transformed into the bacteria. Since the protein was expressed best in Colony 15, it was decided that from this point on we should work with colony 15.

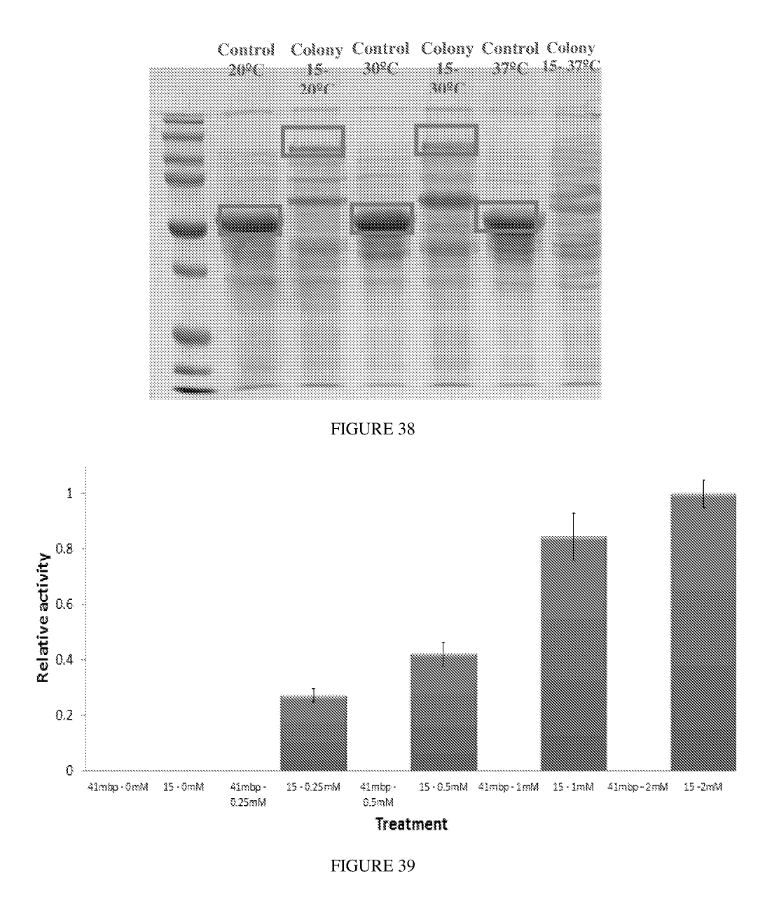

Next, the optimal temperature for the enzyme induction was examined. When comparing between the lysate protein compositions from enzyme induction experiment in different temperatures, it is clear that in 37° C. no considerable induction of the recombinant protein was received while in both 20° C. and 30° C. a considerable induction of the enzyme was received (FIG. 38). In each of the experiments—the control was the lysate from E. Coli BL21 bacterial strain with 41 mbp plasmid.

Copper concentration in induction medium: Analysis of the produced protein activity when the induction was made in the presence of different copper concentrations was made (FIG. 39). In each of the experiments—the control was the lysate from E. Coli BL21 bacterial strain with 41 mbp plasmid. The best lysate activity was received in copper concentration of 2 mM. It was decided to work with this concentration from now on. Enzyme was received in all treatments (FIG. 39).

### Example 7

### Copper Concentration in the Wash and Elution Buffer used in the Protein Purification Process on Amylose Beads Column

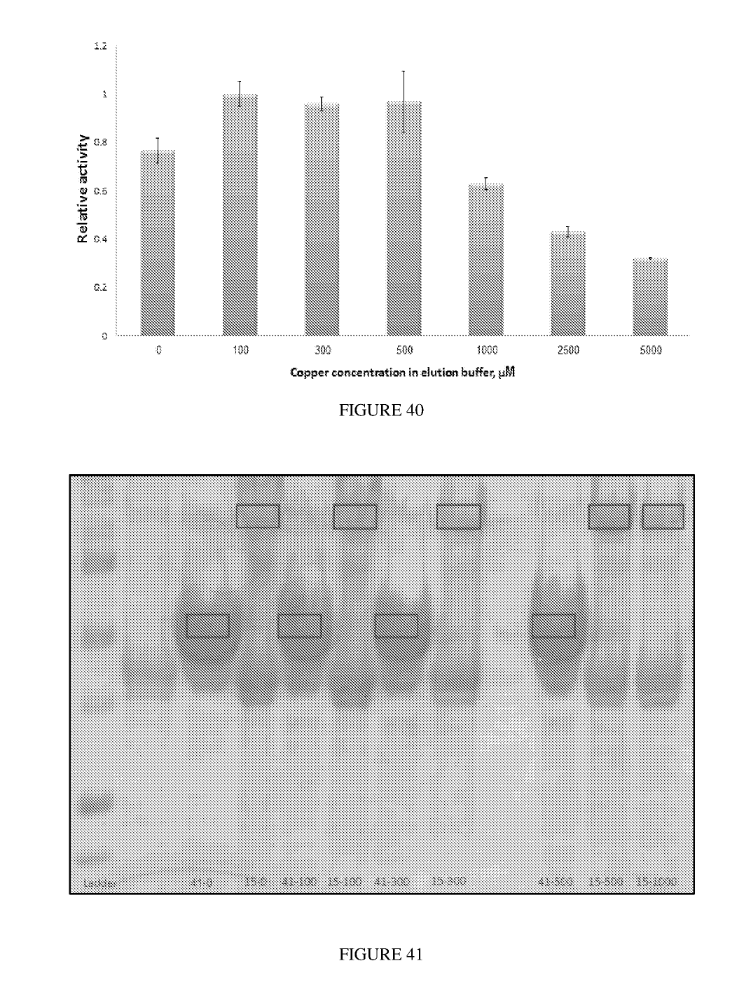

The effect of copper concentration in binding and elution buffer of the purification process was examined (FIGS. 40 and 41). It clearly indicated that a copper concentration of 300 μM copper is optimal for the protein purification processes. All stocks contained protein concentration of 1 mg/mL enzyme.

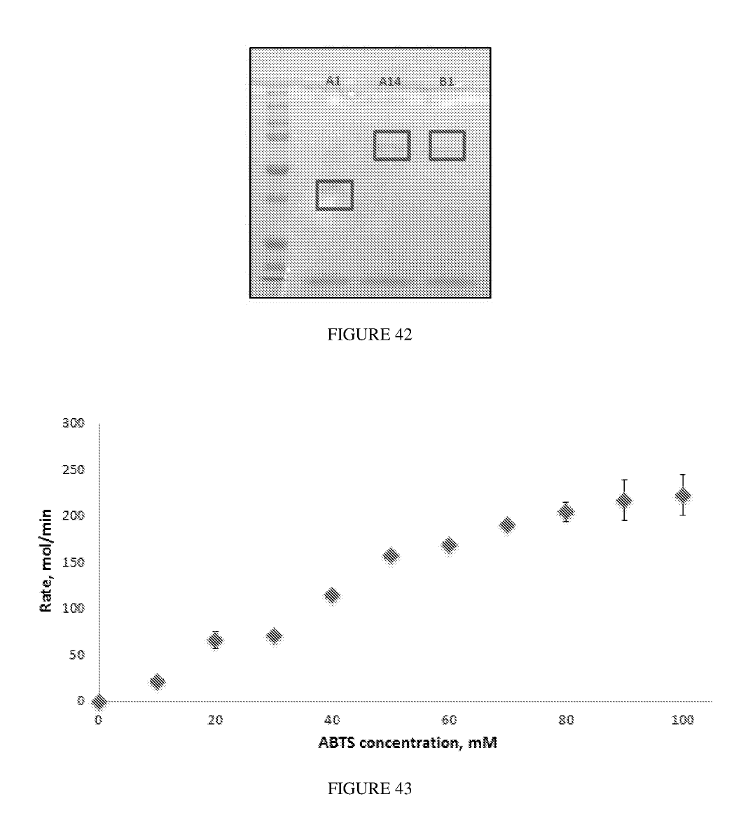

The recombinant enzyme was cleaned upon the amylose beads and cut using Thrombin enzyme. Then it was purified using a mono Q column (strong anion exchanger). The purification process was efficient as two clear peaks were received in 280 nm wave length. An additional peak was received in 330 nm wave length in which maximal absorbance is received by the type III copper in the multi-copper oxidase catalytic site indicates the elution of the enzyme. The different protein fractions were analyzed using 10% SDS page, and indeed a pure protein was received in A14, A15 and B1 fractions (˜60 KDa) and the mbp protein (˜40 KDa) was in the A1 (FIG. 42).

### Example 8

### ABTS Optimal Concentration

The optimal ABTS concentration for the colorimetric reaction was tested (FIG. 43). It was found that the optimal concentration is 80 mM ABTS, and a michaelis menten curve was drawn. The michaelis menten curve indicates that the enzyme Km is 40 mM ABTS, and its Vmax is 220 mol/min.

### Example 9

### The Effect of the Temperature on Purified Enzyme Activity

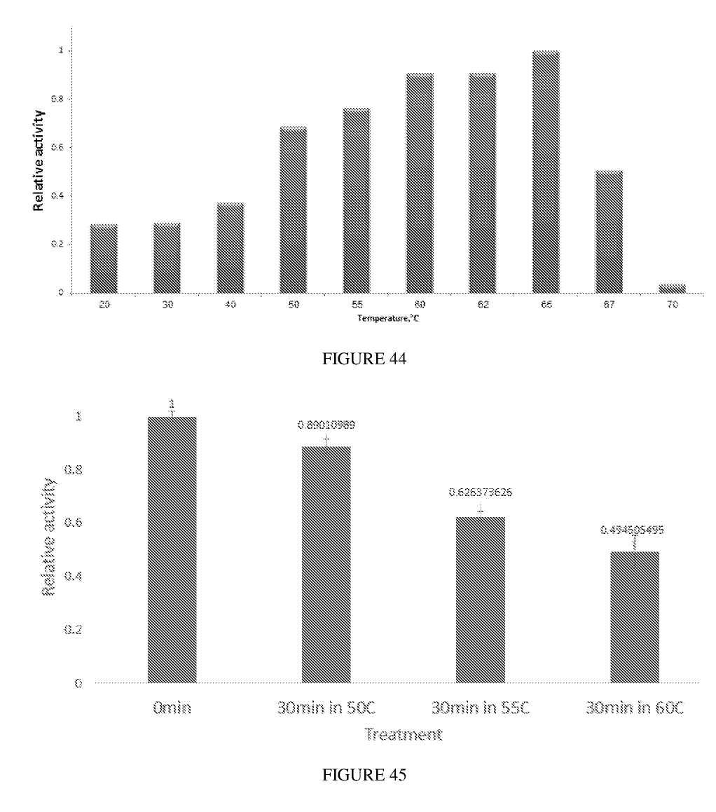

The purified enzyme activity in the presence of 80 mM ABTS was analyzed in different temperature, in the presence of the same enzyme concentration (FIG. 44). It was concluded that 65° C. is the optimal temperature for enzyme activity.

The survival ability of the enzyme was also examined (FIG. 45). The results indicated that pre-incubation of the enzyme in different temperatures prior to its examination led to a decrease in its activity. When comparing the effect of 30 minutes pre-incubation of the enzyme in different temperatures, pre-incubation of the enzyme in 50° C. led to the smallest decrease in the enzyme activity (11%). It should be mentioned that both the survival ability and optimal activity temperature are lower than those of the original enzyme. It is probably because of the bacterium that produced the enzyme.

What is claimed is:

1. A composition comprising: polyethylene and laccase, said laccase has an optimal specific activity at a temperature of 60° C. to 100° C.

2. The composition of claim 1, wherein said composition is maintained at a temperature of 60° C. to 100° C.

3. The composition of claim 1, wherein said laccase comprises the amino acid sequence of SEQ ID NO: 1.

4. The composition of claim 1, wherein said laccase is selected from Brevibacillus borstelensis laccase or Brevibacillus agri laccase.

5. The composition of claim 1, wherein said laccase is an extra-cellular laccase.

6. The composition of claim 1, further comprising Cu²⁺, xylan or both.

7. The composition of claim 1, having a pH of 7.5 to 8.5.

8. A composition comprising: polyethylene, laccase and xylan.

9. The composition of claim 8, comprising Rhodococcus ruber strain C208.

10. The composition of claim 8, wherein said polyethylene is thermo-oxidized.

11. A method for decomposing polyethylene, comprising the step of contacting polyethylene with a laccase having an optimal specific activity at a temperature of 60° C. to 100° C.

12. The method of claim 11, further comprising maintaining said polyethylene and said laccase at a temperature of 60° C. to 100° C.

13. The method of claim 11, further comprising maintaining said polyethylene and said laccase at a pH of 7.5 to 8.5.

14. The method of claim 11, wherein said laccase comprises the amino acid sequence of SEQ ID NO: 1.

15. The method of claim 11, wherein said laccase is selected from Brevibacillus borstelensis laccase or Brevibacillus agri laccase.

16. The method of claim 11, wherein said laccase is an extra-cellular laccase.

17. The method of claim 11, further comprising adding at least one additive selected from Cu²⁺ and xylan to said laccase.

18. A method for decomposing polyethylene, comprising the step of contacting polyethylene with B. borstelensis, B. agri, or a combination of B. borstelensis and B. agri, wherein said polyethylene is the only carbon source for the bacteria.

19. The method of claim 18, further comprising maintaining said polyethylene and said B. borstelensis B. borstelensis, B. agri, or a combination of and B. agri at a temperature of 35° C. to 50° C.

20. The method of claim 18, further comprising maintaining said polyethylene and said B. borstelensis, B. agri, or a combination of B. borstelensis and B. agri at a pH of 7.5 to 8.5.

21. The method of claim 18, further comprising adding at least one additive selected from Cu²⁺ and xylan to said Brevibacillus Agri.

22. A method for decomposing polyethylene, comprising the step of contacting a composition comprising polyethylene and xylan with Rhodococcus ruber strain C208.
